(19) United States
(12) Patent Application Publication
(10) Pub. No.: US 2026/0027295 A1
(43) Pub. Date: Jan. 29, 2026

(54) FLUID INJECTOR SYSTEM WITH IMPROVED RATIO PERFORMANCE

(71) Applicant: BAYER HEALTHCARE LLC, WHIPPANY, NJ (US)

(72) Inventors: Amy Chaya, Strafford, PA (US); Michael Spohn, Fenelton, PA (US); Michael McDermott, Cologne (DE); William Barone, Pittsburgh, PA (US)

(21) Appl. No.: 19/343,381

(22) Filed: Sep. 29, 2025

Related U.S. Application Data
(62) Division of application No. 17/270,616, filed on Feb. 23, 2021, now Pat. No. 12,427,249.

(51) Int. Cl. A61M 5/168; A61M 5/14; A61M 5/142

(52) U.S. Cl. CPC A61M 5/16827; A61M 5/1408; A61M 5/1422; A61M 2005/14208; A61M 2205/52

(57) ABSTRACT

A fluid injector system includes a control device operatively associated with a first drive component configured to pressurize and inject a first fluid and a second drive component configured to pressurize and inject a second fluid. The control device includes at least one processor programmed or configured to: during a first phase of a multi-phase injection protocol, actuate at least the first drive component to inject the first phase; during the first phase of the injection protocol and prior to transitioning to a second phase of the injection protocol, actuate the second drive component to pressurize the second fluid relative to a pressure of the first fluid; and during the second phase of the injection protocol, actuate the second drive component to inject at least the second fluid so that a desired steady-state ratio of the first fluid and the second fluid in the second phase is reached.

### CROSS-REFERENCE TO RELATED APPLICATION

This application is a divisional application of U.S. application Ser. No. 17/270,616, filed Feb. 23, 2021, which is a U.S. national phase application under 35 U.S.C. § 371 of PCT International Application No. PCT/US2019/048276, filed Aug. 27, 2019 and claims the benefit of U.S. Provisional Patent Application No. 62/723,724, filed on Aug. 28, 2018, the disclosure of which is hereby incorporated by reference in its entirety.

### BACKGROUND OF THE DISCLOSURE

### Field of the Disclosure

The present disclosure is related to a fluid injector system and, particularly, to a fluid injector system configured to perform a multi-phase injection protocol. The present disclosure is further related to a fluid injector system having improved ratio performance of a first fluid and a second fluid during a multi-phase injection protocol. The present disclosure is further directed to a method of improving ratio performance of a first fluid and a second fluid during a multi-phase injection protocol using a fluid injector system. The present disclosure is also directed to a computer program product for improving ratio performance of a first and a second fluid during a multi-phase injection protocol using a fluid injector system.

### Description of Related Art

In many medical diagnostic and therapeutic procedures, a medical practitioner, such as a physician or radiologist, injects a patient with one or more fluids. In recent years, a number of injector-actuated syringes and powered injectors for pressurized injection of fluids have been developed for use in procedures such as angiography, computed tomography (CT), molecular imaging (such as PET imaging), and magnetic resonance imaging (MRI). In these procedures, a fluid, such as a contrast agent, may be used to highlight certain internal organs or portions of the body during an imaging process. Meanwhile, saline, or a similar flushing agent, may be used to ensure complete injection of the bolus of the contrast agent or adjust the concentration of the contrast agent.

For fluid injector systems with multi-reservoir disposables set up to deliver more than one fluid type, improving ratio performance of a first fluid and a second fluid during a multi-phase injection protocol is desired. Unintended deviations from desired ratio of a first fluid to second fluid during a simultaneous delivery of both fluids may result in overly concentrated or diluted doses producing images that are non-diagnostic or of reduced quality. Accordingly, there is room for improvement in fluid injector systems, methods of improving ratio performance of a first fluid and a second fluid during a multi-phase injection protocol, and computer program products for use in the same.

### SUMMARY OF THE DISCLOSURE

Accordingly, provided is a fluid injector system configured to perform a multi-phase injection protocol. Also provided is a fluid injector system having improved ratio performance of a first fluid and a second fluid during a multi-phase injection protocol. Also provided is a method of improving ratio performance of a first fluid and a second fluid during a multi-phase injection protocol using a fluid injector system. Also provided is a computer program product for improving ratio performance of a first fluid and a second fluid during a multi-phase injection protocol using a fluid injector system.

In some examples or aspects of the present disclosure, a fluid injector system may be configured to perform an injection protocol including at least a first phase and a second phase, with the second phase being subsequent to the first phase. The fluid injector system may include a control device operatively associated with a first drive component and a second drive component. The first drive component may be configured to pressurize and inject a first fluid from a first fluid reservoir through a fluid conduit. The second drive component may be configured to pressurize and inject a second fluid from a second fluid reservoir through the fluid conduit. The control device may include at least one processor programmed or configured to: during the first phase of the injection protocol, actuate at least the first drive component to inject the first phase through the fluid conduit. The at least one processor may be further programmed or configured to, during the first phase of the injection protocol prior to transitioning to the second phase of the injection protocol, actuate the second drive component to pressurize the second fluid relative to a pressure of the first fluid in the fluid conduit. The at least one processor may be further programmed or configured to, during the second phase of the injection protocol, actuate the second drive component to inject at least the second fluid through the fluid conduit so that a desired steady-state ratio of the first fluid and the second fluid in the second phase is reached.

In some examples or aspects of the present disclosure, the first fluid may include a contrast medium and the second fluid may include a diluent. The desired steady-state ratio of the first fluid and the second fluid in the second phase may be reached at a quicker rate than if the second drive component is not actuated prior to transitioning to the second phase of the injection protocol.

In some examples or aspects of the present disclosure, the at least one processor may be further programmed or configured to, during the first phase of the injection protocol prior to transitioning to the second phase of the injection protocol, cease actuation or reduce a speed of the first drive component. The at least one processor may be further programmed or configured to, during the second phase of the injection protocol, adjust a speed of the first drive component to inject the first fluid at a flow rate to reach the desired steady-state ratio of the first fluid and the second fluid through the fluid conduit.

In some examples or aspects of the present disclosure, the fluid injector system further may include at least a first valve controlling fluid communication between the first reservoir and the fluid conduit and a second valve controlling fluid communication between the second reservoir and the fluid conduit. The at least one processor may be further programmed or configured to close the second valve prior to pressurizing the second fluid relative to the pressure of the first fluid in the fluid conduit. The at least one processor may be further programmed or configured to: open the second valve isolating the second fluid reservoir from the first fluid reservoir and the fluid conduit during a transition from the first phase of the injection protocol to the second phase of the injection protocol.

In some examples or aspects of the present disclosure, pressurizing the second fluid relative to the pressure of the first fluid may include selecting a pressure ratio of the pressure of the first fluid and a pressure of the second fluid to reach the desired steady-state flow ratio of the first fluid and the second fluid for the second phase. Pressurizing the second fluid relative to the pressure of the first fluid may include gradually increasing a pressure of the second fluid such that, at a transition from the first phase of the injection protocol to the second phase of the injection protocol, the pressure of the second fluid is substantially equal to the pressure of the first fluid. Pressurizing the second fluid relative to the pressure of the first fluid may include gradually increasing a pressure of the second fluid such that, at a transition from the first phase of the injection protocol to the second phase of the injection protocol, the pressure of the second fluid is 20% to 150% of the pressure of the first fluid.

In some examples or aspects of the present disclosure, the at least one processor may be further programmed or configured to, during the first phase of the injection protocol prior to transitioning to the second phase of the protocol, adjusting at least one property of the injection protocol to prevent backflow of the first fluid into the second fluid reservoir. Adjusting at least one property of the injection protocol may include at least one of advancing the second drive component at a constant speed, linearly increasing a speed of the second drive component, and exponentially increasing a speed of the second drive component.

In some examples or aspects of the present disclosure, a fluid injector system may include a control device operatively associated with a first drive component and a second drive component, with the first drive component being configured to pressurize and inject a first fluid from a first fluid reservoir through a fluid conduit, and the second drive component being configured to pressurize and inject a second fluid from a second fluid reservoir through the fluid conduit. The control device may include at least one processor programmed or configured to, during the first phase of the injection protocol, actuate at least one of the first drive component and the second drive component to inject at least one of the first fluid and the second fluid through the fluid conduit at a first desired steady-state ratio. The at least one processor may be further programmed or configured to, during an initial portion of the second phase of the injection protocol, actuate the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio. The at least one processor may be further programmed or configured to, subsequent to the initial portion of the second phase, actuate at least one of the first drive component and the second drive component to reduce the volumetric ratio until the volumetric ratio reaches the second desired steady-state ratio of the first fluid and the second fluid in the second phase.

In some examples or aspects of the present disclosure, the first fluid may include a contrast medium and the second fluid may include a diluent. The second desired steady-state ratio of the first fluid and the second fluid in the second phase may be reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceed a second desired steady-state ratio.

In some examples or aspects of the present disclosure, the at least one processor may be further programmed or configured to, during the first phase of the injection protocol prior to transitioning to the second phase of the injection protocol, cease actuation or reduce a speed of the first drive component.

In some examples or aspects of the present disclosure, the fluid injector system further may include at least a first valve controlling fluid communication between the first reservoir and the fluid conduit and a second valve controlling fluid communication between the second reservoir and the fluid conduit. The at least one processor may be further programmed or configured to open the second valve isolating the second fluid reservoir from the first fluid reservoir and the fluid conduit during a transition from the first phase of the injection protocol to the second phase of the injection protocol. The at least one processor may be further programmed or configured to, during the first phase of the injection protocol prior to transitioning to the second phase of the injection protocol, actuate the second drive component to pressurize the second fluid relative to a pressure of the first fluid in the fluid conduit.

In some examples or aspects of the present disclosure, the fluid injector system further may include at least a first valve controlling fluid communication between the first reservoir and the fluid conduit and a second valve controlling fluid communication between the second reservoir and the fluid conduit. The at least one processor may be further programmed or configured to close the second valve prior to pressurizing the second fluid relative to the pressure of the first fluid in the fluid conduit.

In some examples or aspects of the present disclosure, during the first phase of the injection protocol prior to transitioning to the second phase of the injection protocol, at least one processor may be further programmed or configured for adjusting at least one property of the injection protocol to prevent backflow of the first fluid into the second fluid reservoir. Adjusting at least one property of the injection protocol may include at least one of: advancing the second drive component at a constant speed, linearly increasing a speed of the second drive component, and exponentially increasing the speed of the second drive component.

In some examples or aspects of the present disclosure, a computer program product for performing an injection protocol using a fluid injector system may be provided. The computer program product may have non-transitory computer readable media including one or more instructions that, when executed by at least one processor of the fluid injector system, may cause the at least one processor to: during the first phase of the injection protocol, actuate at least a first drive component of the fluid injector system to inject the first phase including a first fluid through a fluid conduit; during the first phase of the injection protocol prior to transitioning to the second phase of the injection protocol, actuate a second drive component of the fluid injector system to pressurize a second fluid relative to a pressure of a first fluid in the fluid conduit; and during the second phase of the injection protocol, actuate the second drive component to inject at least the second fluid through the fluid conduit so that a desired steady-state ratio of the first fluid and the second fluid in the second phase is reached.

In some examples or aspects of the present disclosure, the first fluid may include a contrast medium and the second fluid may include a diluent. The desired steady-state ratio of the first fluid and the second fluid in the second phase may be reached at a quicker rate than if the second drive component is not actuated prior to transitioning to the second phase of the injection protocol.

In some examples or aspects of the present disclosure, the one or more instructions, when executed by the at least one processor of the fluid injector system, further may cause the at least one processor to: during the first phase of the injection protocol prior to transitioning to the second phase of the injection protocol, cease actuation or reduce a speed of the first drive component. The one or more instructions, when executed by at least one processor of the fluid injector system, further may cause the at least one processor to: during the second phase of the injection protocol, adjust a speed of the first drive component to inject the first fluid at a flow rate to reach the desired steady-state ratio of the first fluid and the second fluid through the fluid conduit during the second phase of the injection protocol.

In some examples or aspects of the present disclosure, the fluid injector system further may include at least a first valve controlling fluid communication between the fluid conduit and a first reservoir containing the first fluid, and a second valve controlling fluid communication between the second reservoir and the fluid conduit, and the one or more instructions, when executed by at least one processor of the fluid injector system, further may cause the at least one processor to close the second valve prior to pressurizing the second fluid relative to the pressure of the first fluid in the fluid conduit. The one or more instructions, when executed by at least one processor of the fluid injector system, further may cause the at least one processor to: open the second valve isolating the second fluid reservoir from the first fluid reservoir and the fluid conduit during a transition from the first phase of the injection protocol to the second phase of the injection protocol.

In some examples or aspects of the present disclosure, pressurizing the second fluid relative to the pressure of the first fluid may include selecting a pressure ratio of the pressure of the first fluid and a pressure of the second fluid to reach the desired steady-state flow ratio of the first fluid and the second fluid for the second phase. Pressurizing the second fluid relative to the pressure of the first fluid may include gradually increasing a pressure of the second fluid such that, at a transition from the first phase of the injection protocol to the second phase of the injection protocol, the pressure of the second fluid is substantially equal to the pressure of the first fluid. Pressurizing the second fluid relative to the pressure of the first fluid may include gradually increasing a pressure of the second fluid such that, at a transition from the first phase of the injection protocol to the second phase of the injection protocol, the pressure of the second fluid is 20% to 150% of the pressure of the first fluid.

In some examples or aspects of the present disclosure, the one or more instructions may be further programmed or configured to: during the first phase of the injection protocol prior to transitioning to the second phase of the injection protocol, adjusting at least one property of the injection protocol to prevent backflow of the first fluid into the second fluid reservoir. Adjusting at least one property of the injection protocol may include at least one of: advancing the second drive component at a constant speed, linearly increasing a speed of the second drive component, and exponentially increasing the speed of the second drive component.

In some examples or aspects of the present disclosure, a computer program product may be configured to perform an injection protocol using a fluid injector system. The injection protocol may include at least a first phase and a second phase subsequent to the first phase. The computer program product may include non-transitory computer readable media including one or more instructions that, when executed by at least one processor of the fluid injector system, may cause the at least one processor to: during the first phase of the injection protocol, actuate at least one of a first drive component, configured to pressurize and inject a first fluid from a first fluid reservoir through a fluid conduit and a second drive component, configured to pressurize and inject a second fluid from a second fluid reservoir through the fluid conduit, to inject at least one of the first fluid and the second fluid through the fluid conduit at a first desired steady-state ratio; during an initial portion of the second phase of the injection protocol, actuate the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio; and subsequent to the initial portion of the second phase, actuate at least one of the first drive component and the second drive component to reduce the volumetric ratio until the volumetric ratio reaches the second desired steady-state ratio of the first fluid and the second fluid in the second phase.

In some examples or aspects of the present disclosure, the first fluid may include a contrast medium and the second fluid may include a diluent. The second desired steady-state ratio of the first fluid and the second fluid in the second phase may be reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceed a second desired steady-state ratio.

In some examples or aspects of the present disclosure, the one or more instructions, when executed by at least one processor of the fluid injector system, further may cause the at least one processor to: during the first phase of the injection protocol prior to transitioning to the second phase of the injection protocol, cease actuation or reduce a speed of the first drive component.

In some examples or aspects of the present disclosure, the fluid injector system further may include at least a first valve controlling fluid communication between the first reservoir and the fluid conduit and a second valve controlling fluid communication between the second reservoir and the fluid conduit, and the one or more instructions, when executed by at least one processor of the fluid injector system, further may cause the at least one processor to: open the second valve isolating the second drive component from the first fluid reservoir and the fluid conduit during a transition from the first phase of the injection protocol to the second phase of the injection protocol. The one or more instructions, when executed by at least one processor of the fluid injector system, further may cause the at least one processor to: during the first phase of the injection protocol prior to transitioning to the second phase of the injection protocol, actuate the second drive component to pressurize the second fluid relative to a pressure of the first fluid in the fluid conduit.

In some examples or aspects of the present disclosure, the fluid injector system further may include at least a first valve controlling fluid communication between the first reservoir and the fluid conduit and a second valve controlling fluid communication between the second reservoir and the fluid conduit, and the one or more instructions, when executed by at least one processor of the fluid injector system, further may cause the at least one processor to close the second valve prior to pressurizing the second fluid relative to the pressure of the first fluid in the fluid conduit.

In some examples or aspects of the present disclosure, the one or more instructions, when executed by at least one processor of the fluid injector system, further may cause the at least one processor to, during the first phase of the injection protocol prior to transitioning to the second phase of the injection protocol, adjust at least one property of the injection protocol to prevent backflow of the first fluid into the second fluid reservoir. Adjusting at least one property of the injection protocol may include at least one of: advancing the second drive component at a constant speed, linearly increasing a speed of the second drive component, and exponentially increasing the speed of the second drive component.

In some examples or aspects of the present disclosure, a method of performing an injection protocol including at least a first phase and a second phase using a fluid injector system may include: providing a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, actuating at least the first drive component to inject the first phase through the fluid conduit during the first phase of the injection protocol; actuating the second drive component to pressurize the second fluid relative to a pressure of the first fluid in the fluid conduit during the first phase of the injection protocol prior to transitioning to the second phase of the injection protocol; and actuating the second drive component to inject at least the second fluid through the fluid conduit so that a desired steady-state ratio of the first fluid and the second fluid in the second phase is reached during the second phase of the injection protocol.

In some examples or aspects of the present disclosure, the first fluid may include a contrast medium and the second fluid may include a diluent. The desired steady-state ratio of the first fluid and the second fluid in the second phase may be reached at a quicker rate than if the second drive component is not actuated prior to transitioning to the second phase of the injection protocol.

In some examples or aspects of the present disclosure, the method further may include ceasing actuation or reducing a speed of the first drive component during the first phase of the injection protocol prior to transitioning to the second phase of the injection protocol. The method further may include adjusting a speed of the first drive component to inject the first fluid at a flow rate to reach the desired steady-state ratio of the first fluid and the second fluid through the fluid conduit during the second phase of the injection protocol.

In some examples or aspects of the present disclosure, the fluid injector system may further include at least a first valve controlling fluid communication between the first reservoir and the fluid conduit and a second valve controlling fluid communication between the second reservoir and the fluid conduit, and the method further may include closing the second valve prior to pressurizing the second fluid relative to the pressure of the first fluid in the fluid conduit. The method further may include opening the second valve isolating the second fluid reservoir from the first fluid reservoir and the fluid conduit during a transition from the first phase of the injection protocol to the second phase of the injection protocol.

In some examples or aspects of the present disclosure, pressurizing the second fluid relative to the pressure of the first fluid may include selecting a pressure ratio of the pressure of the first fluid and a pressure of the second fluid to reach the desired steady-state flow ratio of the first fluid and the second fluid for the second phase. Pressurizing the second fluid relative to the pressure of the first fluid may include gradually increasing a pressure of the second fluid such that, at a transition from the first phase of the injection protocol to the second phase of the injection protocol, the pressure of the second fluid is substantially equal to the pressure of the first fluid. Pressurizing the second fluid relative to the pressure of the first fluid may include gradually increasing a pressure of the second fluid such that, at a transition from the first phase of the injection protocol to the second phase of the injection protocol, the pressure of the second fluid is 20% to 150% of the pressure of the first fluid.

In some examples or aspects of the present disclosure, the method further may include adjusting at least one property of the injection protocol to prevent backflow of the first fluid into the second fluid reservoir during the first phase of the injection protocol prior to transitioning to the second phase of the injection protocol. Adjusting at least one property of the injection protocol may include at least one of: advancing the second drive component at a constant speed, linearly increasing a speed of the second drive component, and exponentially increasing the speed of the second drive component.

In some examples or aspects of the present disclosure, a method of performing an injection protocol including at least a first phase and a second phase using a fluid injector system may include: providing a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, actuating at least one of the first drive component and the second drive component to inject at least one of the first fluid and the second fluid through the fluid conduit at a first desired steady-state ratio during the first phase of the injection protocol; actuating the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio during an initial portion of the second phase of the injection protocol; and actuating at least one of the first drive component and the second drive component to reduce the volumetric ratio until the volumetric ratio reaches the second desired steady-state ratio of the first fluid and the second fluid in the second phase subsequent to the initial portion of the second phase.

In some examples or aspects of the present disclosure, the first fluid may include a contrast medium and the second fluid may include a diluent. The second desired steady-state ratio of the first fluid and the second fluid in the second phase may be reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceed a second desired steady-state ratio.

In some examples or aspects of the present disclosure, the method further may include ceasing actuation or reduce a speed of the first drive component during the first phase of the injection protocol prior to transitioning to the second phase of the injection protocol. The fluid injector system further may include at least a first valve controlling fluid communication between the first reservoir and the fluid conduit and a second valve controlling fluid communication between the second reservoir and the fluid conduit, and the method further may include opening the second valve isolating the second drive component from the first fluid reservoir and the fluid conduit during a transition from the first phase of the injection protocol to the second phase of the injection protocol.

In some examples or aspects of the present disclosure, the method further may include actuating the second drive component to pressurize the second fluid relative to a pressure of the first fluid in the fluid conduit during the first phase of the injection protocol prior to transitioning to the second phase of the injection protocol. The fluid injector system further may include at least a first valve controlling fluid communication between the first reservoir and the fluid conduit and a second valve controlling fluid communication between the second reservoir and the fluid conduit, and the method further may include closing the second valve prior to pressurizing the second fluid relative to the pressure of the first fluid in the fluid conduit.

In some examples or aspects of the present disclosure, the method further may include adjusting at least one property of the injection protocol to prevent backflow of the first fluid into the second fluid reservoir during the first phase of the injection protocol prior to transitioning to the second phase of the injection protocol. Adjusting at least one property of the injection protocol may include at least one of: advancing the second drive component at a constant speed, linearly increasing a speed of the second drive component, and exponentially increasing the speed of the second drive component.

Various other aspects of the present disclosure are recited in one or more of the following numbered clauses:
Clause 1: A fluid injector system configured to perform an injection protocol comprising at least a first phase and a second phase, the second phase subsequent to the first phase, the fluid injector system comprising: a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, wherein the control device comprises at least one processor programmed or configured to: during the first phase of the injection protocol, actuate at least the first drive component to inject the first phase through the fluid conduit; during the first phase of the injection protocol prior to transitioning to the second phase of the injection protocol, actuate the second drive component to pressurize the second fluid relative to a pressure of the first fluid in the fluid conduit; and during the second phase of the injection protocol, actuate the second drive component to inject at least the second fluid through the fluid conduit so that a desired steady-state ratio of the first fluid and the second fluid in the second phase is reached.
Clause 2: The fluid injector system of clause 1, wherein the first fluid comprises a contrast medium and the second fluid comprises a diluent.
Clause 3: The fluid injector system of clause 1 or 2, wherein the desired steady-state ratio of the first fluid and the second fluid in the second phase is reached at a quicker rate than if the second drive component is not actuated prior to transitioning to the second phase of the injection protocol.
Clause 4: The fluid injector system of any of clauses 1 to 3, wherein the at least one processor is further programmed or configured to: during the first phase of the injection protocol prior to transitioning to the second phase of the injection protocol, cease actuation or reduce a speed of the first drive component.
Clause 5: The fluid injector system of any of clauses 1 to 4, wherein the at least one processor is further programmed or configured to: during the second phase of the injection protocol, adjust a speed of the first drive component to inject the first fluid at a flow rate to reach a desired steady-state ratio of the first and second fluid through the fluid conduit.
Clause 6: The fluid injector system of any of clauses 1 to 5, further comprising at least a first valve controlling fluid communication between the first reservoir and the fluid conduit and a second valve controlling fluid communication between the second reservoir and the fluid conduit, wherein the at least one processor is further programmed or configured to close the second valve prior to pressurizing the second fluid relative to the pressure of the first fluid in the fluid conduit.
Clause 7: The fluid injector system of any of clauses 1 to 6, wherein the at least one processor is further programmed or configured to: open the second valve isolating the second fluid reservoir from the first fluid reservoir and the fluid conduit during a transition from the first phase of the injection protocol to the second phase of the injection protocol.
Clause 8: The fluid injector system of any of clauses 1 to 7, wherein pressurizing the second fluid relative to the pressure of the first fluid comprises selecting a pressure ratio of the pressure of the first fluid and a pressure of the second fluid to reach the desired steady-state flow ratio of the first fluid and the second fluid for the second phase.
Clause 9: The fluid injector system of any of clauses 1 to 8, wherein pressurizing the second fluid relative to the pressure of the first fluid comprises gradually increasing a pressure of the second fluid such that at a transition from the first phase of the injection protocol to the second phase of the injection protocol, the pressure of the second fluid is substantially equal to the pressure of the first fluid. The fluid injector system of any of clauses 1 to 8, wherein pressurizing the second fluid relative to the pressure of the first fluid comprises gradually increasing a pressure of the second fluid such that at a transition from the first phase of the injection protocol to the second phase of the injection protocol, the pressure of the second fluid is 20% to 150% of the pressure of the first fluid.
Clause 10: The fluid injector system of any of clauses 1 to 9, wherein the at least one processor is further programmed or configured to: during the first phase of the injection protocol prior to transitioning to the second phase of the injection protocol, adjusting at least one property of the injection protocol to prevent backflow of the first fluid into the second fluid reservoir.
Clause 11: The fluid injector system of any of clauses 1 to 10, wherein adjusting at least one property of the injection protocol comprises at least one of: advancing the second drive component at a constant speed, linearly increasing a speed of the second drive component, and exponentially increasing the speed of the second drive component.
Clause 12: A fluid injector system configured to perform an injection protocol comprising at least a first phase and a second phase, the second phase subsequent to the first phase, the fluid injector system comprising: a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, wherein the control device comprises at least one processor programmed or configured to: during the first phase of the injection protocol, actuate at least one of the first drive component and the second drive component to inject at least one of the first fluid and the second fluid through the fluid conduit at a first desired steady-state ratio; during an initial portion of the second phase of the injection protocol, actuate the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio; and subsequent to the initial portion of the second phase, actuate at least one of the first drive component and the second drive component to reduce the volumetric ratio until the volumetric ratio reaches the second desired steady-state ratio of the first fluid and the second fluid in the second phase.
Clause 13: The fluid injector system of clause 12, wherein the first fluid comprises a contrast medium and the second fluid comprises a diluent.
Clause 14: The fluid injector system of clause 12 or 13, wherein the second desired steady-state ratio of the first fluid and the second fluid in the second phase is reached at a quicker rate than if the actuation of the first drive component and the second drive component to inject the first and second fluid through the fluid conduit such that a volumetric ratio of the second fluid displaced from the second fluid reservoir relative to the first fluid displaced from the first fluid reservoir does not exceed a second desired steady-state ratio.
Clause 15: The fluid injector system of any of clauses 12 to 14, wherein the at least one processor is further programmed or configured to: during the first phase of the injection protocol prior to transitioning to the second phase of the injection protocol, cease actuation or reduce a speed of the first drive component.
Clause 16: The fluid injector system of any of clauses 12 to 15, further comprising at least a first valve controlling fluid communication between the first reservoir and the fluid conduit and a second valve controlling fluid communication between the second reservoir and the fluid conduit, wherein the at least one processor is further programmed or configured to: open the second valve isolating the second drive component from the first fluid reservoir and the fluid conduit during a transition from the first phase of the injection protocol to the second phase of the injection protocol.
Clause 17: The fluid injector system of any of clauses 12 to 16, wherein the at least one processor is further programmed or configured to: during the first phase of the injection protocol prior to transitioning to the second phase of the injection protocol, actuate the second drive component to pressurize the second fluid relative to a pressure of the first fluid in the fluid conduit.
Clause 18: The fluid injector system of any of clauses 12 to 17, further comprising at least a first valve controlling fluid communication between the first reservoir and the fluid conduit and a second valve controlling fluid communication between the second reservoir and the fluid conduit, wherein the at least one processor is further programmed or configured to close the second valve prior to pressurizing the second fluid relative to the pressure of the first fluid in the fluid conduit.
Clause 19: The fluid injector system of any of clauses 12 to 18, wherein the at least one processor is further programmed or configured to: during the first phase of the injection protocol prior to transitioning to the second phase of the injection protocol, adjust at least one property of the injection protocol to prevent backflow of the first fluid into the second fluid reservoir.
Clause 20: The fluid injector system of any of clauses 12 to 19, wherein adjusting at least one property of the injection protocol comprises at least one of: advancing the second drive component at a constant speed, linearly increasing a speed of the second drive component, and exponentially increasing the speed of the second drive component.
Clause 21: A computer program product for performing an injection protocol using a fluid injector system, the injection protocol including at least a first phase and a second phase subsequent to the first phase, the computer program product comprising: non-transitory computer readable media comprising one or more instructions that, when executed by at least one processor of the fluid injector system, cause the at least one processor to: during the first phase of the injection protocol, actuate at least a first drive component of the fluid injector system to inject the first phase including a first fluid through a fluid conduit; during the first phase of the injection protocol prior to transitioning to the second phase of the injection protocol, actuate a second drive component of the fluid injector system to pressurize a second fluid relative to a pressure of a first fluid in the fluid conduit; and during the second phase of the injection protocol, actuate the second drive component to inject at least the second fluid through the fluid conduit so that a desired steady-state ratio of the first fluid and the second fluid in the second phase is reached.
Clause 22: The computer program product of clause 21, wherein the first fluid comprises a contrast medium and the second fluid comprises a diluent.
Clause 23: The computer program product of clause 21 or 22, wherein the desired steady-state ratio of the first fluid and the second fluid in the second phase is reached at a quicker rate than if the second drive component is not actuated prior to transitioning to the second phase of the injection protocol.
Clause 24: The computer program product of any of clauses 21 to 23, wherein the one or more instructions, when executed by the at least one processor of the fluid injector system, further cause the at least one processor to: during the first phase of the injection protocol prior to transitioning to the second phase of the injection protocol, cease actuation or reduce a speed of the first drive component.
Clause 25: The computer program product of any of clauses 21 to 24, wherein the one or more instructions, when executed by at least one processor of the fluid injector system, further cause the at least one processor to: during the second phase of the injection protocol, adjust a speed of the first drive component to inject the first fluid at a flow rate to reach the desired steady-state ratio of the first fluid and the second fluid through the fluid conduit during the second phase of the injection protocol.
Clause 26: The computer program product of any of clauses 21 to 25, wherein the fluid injector system further comprises at least a first valve controlling fluid communication between the fluid conduit and a first reservoir containing the first fluid, and a second valve controlling fluid communication between the second reservoir and the fluid conduit, and wherein the one or more instructions, when executed by at least one processor of the fluid injector system, further cause the at least one processor to close the second valve prior to pressurizing the second fluid relative to the pressure of the first fluid in the fluid conduit.
Clause 27: The computer program product of clause 26, wherein the one or more instructions, when executed by at least one processor of the fluid injector system, further cause the at least one processor to: open the second valve isolating the second fluid reservoir from the first fluid reservoir and the fluid conduit during a transition from the first phase of the injection protocol to the second phase of the injection protocol.
Clause 28: The computer program product of any of clauses 21 to 27, wherein pressurizing the second fluid relative to the pressure of the first fluid comprises selecting a pressure ratio of the pressure of the first fluid and a pressure of the second fluid to reach the desired steady-state flow ratio of the first fluid and the second fluid for the second phase.
Clause 29: The computer program product of any of clauses 21 to 28, wherein pressurizing the second fluid relative to the pressure of the first fluid comprises gradually increasing a pressure of the second fluid such that, at a transition from the first phase of the injection protocol to the second phase of the injection protocol, the pressure of the second fluid is substantially equal to the pressure of the first fluid. The computer program product of any of clauses 21 to 28, wherein pressurizing the second fluid relative to the pressure of the first fluid comprises gradually increasing a pressure of the second fluid such that at a transition from the first phase of the injection protocol to the second phase of the injection protocol, the pressure of the second fluid is 20% to 150% of the pressure of the first fluid.
Clause 30: The computer program product of any of clauses 21 to 29, wherein the at least one processor is further programmed or configured to: during the first phase of the injection protocol prior to transitioning to the second phase of the injection protocol, adjusting at least one property of the injection protocol to prevent backflow of the first fluid into the second fluid reservoir.
Clause 31: The computer program product of clause 30, wherein adjusting at least one property of the injection protocol comprises at least one of: advancing the second drive component at a constant speed, linearly increasing a speed of the second drive component, and exponentially increasing the speed of the second drive component.
Clause 32: A computer program product configured to perform an injection protocol using a fluid injector system, the injection protocol including at least a first phase and a second phase subsequent to the first phase, the computer program product comprising: non-transitory computer readable media comprising one or more instructions that, when executed by at least one processor of the fluid injector system, cause the at least one processor to: during the first phase of the injection protocol, actuate at least one of a first drive component, configured to pressurize and inject a first fluid from a first fluid reservoir through a fluid conduit and a second drive component, configured to pressurize and inject a second fluid from a second fluid reservoir through the fluid conduit, to inject at least one of the first fluid and the second fluid through the fluid conduit at a first desired steady-state ratio; during an initial portion of the second phase of the injection protocol, actuate the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio; and subsequent to the initial portion of the second phase, actuate at least one of the first drive component and the second drive component to reduce the volumetric ratio until the volumetric ratio reaches the second desired steady-state ratio of the first fluid and the second fluid in the second phase.
Clause 33: The computer program product of clause 32, wherein the first fluid comprises a contrast medium and the second fluid comprises a diluent.
Clause 34: The computer program product of clause 32 or 33, wherein the second desired steady-state ratio of the first fluid and the second fluid in the second phase is reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceeds a second desired steady-state ratio.
Clause 35: The computer program product of any of clauses 32 to 34, wherein the one or more instructions, when executed by at least one processor of the fluid injector system, further cause the at least one processor to: during the first phase of the injection protocol prior to transitioning to the second phase of the injection protocol, cease actuation or reduce a speed of the first drive component.
Clause 36: The computer program product of any of clauses 32 to 35, wherein the fluid injector system further comprises at least a first valve controlling fluid communication between the first reservoir and the fluid conduit and a second valve controlling fluid communication between the second reservoir and the fluid conduit, and wherein the one or more instructions, when executed by at least one processor of the fluid injector system, further cause the at least one processor to: open the second valve isolating the second drive component from the first fluid reservoir and the fluid conduit during a transition from the first phase of the injection protocol to the second phase of the injection protocol.
Clause 37: The computer program product of any of clauses 32 to 36, wherein the one or more instructions, when executed by at least one processor of the fluid injector system, further cause the at least one processor to: during the first phase of the injection protocol prior to transitioning to the second phase of the injection protocol, actuate the second drive component to pressurize the second fluid relative to a pressure of the first fluid in the fluid conduit.
Clause 38: The computer program product of clause 37, wherein the fluid injector system further comprises at least a first valve controlling fluid communication between the first reservoir and the fluid conduit and a second valve controlling fluid communication between the second reservoir and the fluid conduit, and wherein the one or more instructions, when executed by at least one processor of the fluid injector system, further cause the at least one processor to close the second valve prior to pressurizing the second fluid relative to the pressure of the first fluid in the fluid conduit.
Clause 39: The computer program product of any of clauses 32 to 38, wherein the one or more instructions, when executed by at least one processor of the fluid injector system, further cause the at least one processor to, during the first phase of the injection protocol prior to transitioning to the second phase of the injection protocol, adjust at least one property of the injection protocol to prevent backflow of the first fluid into the second fluid reservoir.
Clause 40: The computer program product of any of clauses 32 to 39, wherein adjusting at least one property of the injection protocol comprises at least one of: advancing the second drive component at a constant speed, linearly increasing a speed of the second drive component, and exponentially increasing the speed of the second drive component.
Clause 41: A method of performing an injection protocol comprising at least a first phase and a second phase using a fluid injector system, the second phase subsequent to the first phase, the method comprising: providing a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, actuating at least the first drive component to inject the first phase through the fluid conduit during the first phase of the injection protocol; actuating the second drive component to pressurize the second fluid relative to a pressure of the first fluid in the fluid conduit during the first phase of the injection protocol prior to transitioning to the second phase of the injection protocol; and actuating the second drive component to inject at least the second fluid through the fluid conduit so that a desired steady-state ratio of the first fluid and the second fluid in the second phase is reached during the second phase of the injection protocol.
Clause 42: The method of clause 41, wherein the first fluid comprises a contrast medium and the second fluid comprises a diluent.
Clause 43: The method of clause 41 or 42, wherein the desired steady-state ratio of the first fluid and the second fluid in the second phase is reached at a quicker rate than if the second drive component is not actuated prior to transitioning to the second phase of the injection protocol.
Clause 44: The method of any of clauses 41 to 43, further comprising ceasing actuation or reducing a speed of the first drive component during the first phase of the injection protocol prior to transitioning to the second phase of the injection protocol.
Clause 45: The method of any of clauses 41 to 44, further comprising adjusting a speed of the first drive component to inject the first fluid at a flow rate to reach the desired steady-state ratio of the first fluid and the second fluid through the fluid conduit during the second phase of the injection protocol.
Clause 46: The method of any of clauses 41 to 45, wherein the fluid injector system comprises at least a first valve controlling fluid communication between the first reservoir and the fluid conduit and a second valve controlling fluid communication between the second reservoir and the fluid conduit, and wherein the method further comprises closing the second valve prior to pressurizing the second fluid relative to the pressure of the first fluid in the fluid conduit.
Clause 47: The method of clause 46, further comprising opening the second valve isolating the second fluid reservoir from the first fluid reservoir and the fluid conduit during a transition from the first phase of the injection protocol to the second phase of the injection protocol.
Clause 48: The method of any of clauses 41 to 47, wherein pressurizing the second fluid relative to the pressure of the first fluid comprises selecting a pressure ratio of the pressure of the first fluid and a pressure of the second fluid to reach the desired steady-state flow ratio of the first fluid and the second fluid for the second phase.
Clause 49: The method of any of clauses 41 to 48, wherein pressurizing the second fluid relative to the pressure of the first fluid comprises gradually increasing a pressure of the second fluid such that, at a transition from the first phase of the injection protocol to the second phase of the injection protocol, the pressure of the second fluid is substantially equal to the pressure of the first fluid. The method of any of clauses 41 to 48, wherein pressurizing the second fluid relative to the pressure of the first fluid comprises gradually increasing a pressure of the second fluid such that at a transition from the first phase of the injection protocol to the second phase of the injection protocol, the pressure of the second fluid is 20% to 150% of the pressure of the first fluid.
Clause 50: The method of any of clauses 41 to 49, further comprising adjusting at least one property of the injection protocol to prevent backflow of the first fluid into the second fluid reservoir during the first phase of the injection protocol prior to transitioning to the second phase of the injection protocol.
Clause 51: The method of clause 50, wherein adjusting at least one property of the injection protocol comprises at least one of: advancing the second drive component at a constant speed, linearly increasing a speed of the second drive component, and exponentially increasing the speed of the second drive component.
Clause 52: A method of performing an injection protocol comprising at least a first phase and a second phase using a fluid injector system, the second phase subsequent to the first phase, the method comprising: providing a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit, actuating at least one of the first drive component and the second drive component to inject at least one of the first fluid and the second fluid through the fluid conduit at a first desired steady-state ratio during the first phase of the injection protocol; actuating the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio during an initial portion of the second phase of the injection protocol; and actuating at least one of the first drive component and the second drive component to reduce the volumetric ratio until the volumetric ratio reaches the second desired steady-state ratio of the first fluid and the second fluid in the second phase subsequent to the initial portion of the second phase.
Clause 53: The method of clause 52, wherein the first fluid comprises a contrast medium and the second fluid comprises a diluent.
Clause 54: The method of clause 52 or 53, wherein the second desired steady-state ratio of the first fluid and the second fluid in the second phase is reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceed a second desired steady-state ratio.
Clause 55: The method of any of clauses 52 to 54, further comprising ceasing actuation or reduce a speed of the first drive component during the first phase of the injection protocol prior to transitioning to the second phase of the injection protocol.
Clause 56: The method of any of clauses 52 to 55, wherein the fluid injector system further comprises at least a first valve controlling fluid communication between the first reservoir and the fluid conduit and a second valve controlling fluid communication between the second reservoir and the fluid conduit, and wherein the method further comprises opening the second valve isolating the second drive component from the first fluid reservoir and the fluid conduit during a transition from the first phase of the injection protocol to the second phase of the injection protocol.
Clause 57: The method of any of clauses 52 to 56, further comprising actuating the second drive component to pressurize the second fluid relative to a pressure of the first fluid in the fluid conduit during the first phase of the injection protocol prior to transitioning to the second phase of the injection protocol.
Clause 58: The method of clause 57, wherein the fluid injector system further comprises at least a first valve controlling fluid communication between the first reservoir and the fluid conduit and a second valve controlling fluid communication between the second reservoir and the fluid conduit, and wherein the method further comprises closing the second valve prior to pressurizing the second fluid relative to the pressure of the first fluid in the fluid conduit.
Clause 59: The method of any of clauses 52 to 58, further comprising adjusting at least one property of the injection protocol to prevent backflow of the first fluid into the second fluid reservoir during the first phase of the injection protocol prior to transitioning to the second phase of the injection protocol.
Clause 60: The method of clause 59, wherein adjusting at least one property of the injection protocol comprises at least one of: advancing the second drive component at a constant speed, linearly increasing a speed of the second drive component, and exponentially increasing the speed of the second drive component.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a fluid injector system according to one example of the present disclosure;

FIG. 2 is a perspective view of a multi-use disposable set for use with a fluid injector system of FIG. 1;

FIG. 3 is a perspective view of a fluid injector system according to another example of the present disclosure;

FIG. 4 is a schematic view of an electronic control system of a fluid injector system in accordance with examples of the present disclosure;

FIG. 5 is a graph comparing actual fluid delivery of prior art fluid injector systems to actual fluid delivery of a fluid injector system according to the present disclosure, for an injector protocol having a first phase of 100% contrast and a second phase of 95% contrast to 5% saline;

FIG. 6 is a graph comparing actual fluid delivery of prior art fluid injector systems to actual fluid delivery of a fluid injector system according to the present disclosure, for an injector protocol having a first phase of 100% contrast and a second phase of 75% contrast to 25% saline;

FIG. 7 is a graph comparing actual fluid delivery of prior art fluid injector systems to actual fluid delivery of a fluid injector system according to the present disclosure, for an injector protocol having a first phase of 100% contrast and a second phase of 50% contrast to 50% saline;

FIG. 8 is a graph comparing actual fluid delivery of prior art fluid injector systems to actual fluid delivery of a fluid injector system according to the present disclosure, for an injector protocol having a first phase of 100% contrast and a second phase of 25% contrast to 75% saline;

FIG. 9 is a graph showing a relationship of a drive speed profile of a first drive component and a second drive component as a function of time according to one example of the present disclosure;

FIG. 10 is a graph showing a relationship of a drive profile of a first drive component and a second drive component as a function of time according to another example of the present disclosure;

FIG. 11 is a graph showing a relationship of a drive profile of a first drive component and a second drive component as a function of time according to another example of the present disclosure;

FIG. 12 is a step sequence diagram of a method of improving ratio performance of a first fluid and a second fluid during a multi-phase injection protocol using a fluid injector system according to one example of the present disclosure; and

FIG. 13 is a step sequence diagram of a method of improving ratio performance of a first fluid and a second fluid during a multi-phase injection protocol using a fluid injector system according to another example of the present disclosure.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a multi-patient disposable set, the term “proximal” refers to a portion of a syringe nearest a piston for delivering fluid from a syringe.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a fluid reservoir, such as a syringe, a rolling diaphragm, or multiple syringe disposable set, the term “distal” refers to a portion of the fluid reservoir nearest to a patient. When used in relation to a fluid reservoir, such as a syringe, a rolling diaphragm, or multiple syringe disposable set, the term “proximal” refers to a portion of the fluid reservoir nearest to the injector system.

The term “open”, when used to refer to a fluid delivery component, means that the fluid reservoir is in fluid connection with an outlet to atmospheric pressure or connected to a patient's vascular system, for example through a nozzle or the open end of a tubing component or catheter. In an open system, fluid flow may be constrained or restricted, for example by forcing a fluid through a small diameter fluid path where flow may be determined by physical parameters of the system and the fluid, such as tubing diameter, fluid path constrictions, applied pressure, viscosity, etc. The term “closed” or “closeable”, when used to refer to a fluid delivery component, means that the fluid reservoir has at least one state in which the component is not in fluid connection with an outlet under atmospheric pressure or connected to a patient's vascular system or the fluid in the fluid reservoir is fluidly isolated, for example where fluid flow is stopped by a valve, such as a stopcock, high crack pressure valve, pinch valve, and the like, that closes a fluid pathway.

As used herein, the terms “capacitance” and “impedance” are used interchangeably to refer to a volumetric expansion of fluid reservoirs, syringes, fluid lines, and/or other components of a fluid delivery system as a result of pressurized fluids with such components. Capacitance and impedance may be due to high injection pressures, which may be on the order of 1,200 psi in some angiographic procedures, and may result in a volume of fluid in excess of the desired quantity selected for the injection procedure.

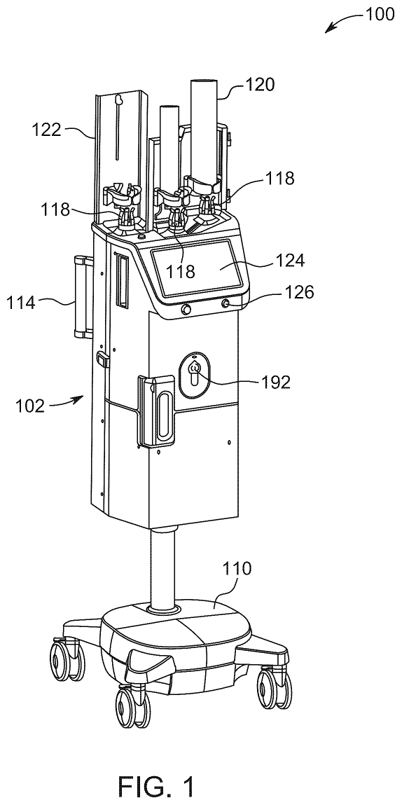

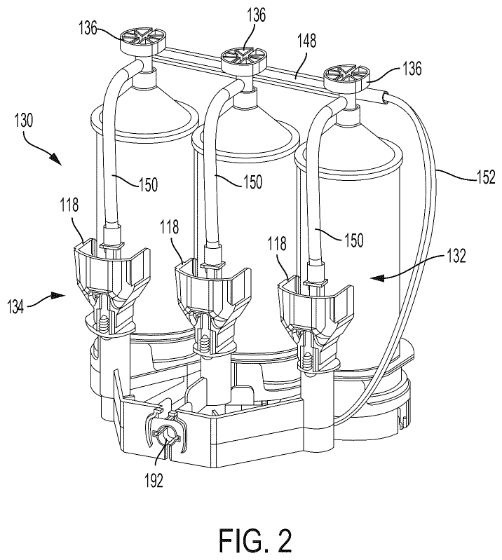

Referring to the drawings in which like reference characters refer to like parts throughout the several views thereof, one aspect or example of the present disclosure is generally directed to a multi-fluid medical injector/injector system 100 (hereinafter “fluid injector system 100”) which in certain embodiments may include a multi-use disposable set (MUDS) 130 configured for delivering fluid to a patient using a single-use disposable set (SUDS) 190 connector and in other embodiments may include two or more disposable fluid reservoirs or syringes, which may be disposed after one injection procedure or a specific number of injection procedures. The fluid injector system 100 may include multiple components as individually described herein. Generally, the fluid injector system 100 depicted in FIGS. 1-2 has a powered injector or other administration device and a fluid delivery set intended to be associated with the injector to deliver one or more fluids from one or more multi-dose containers under pressure into a patient, as described herein. The various devices, components, and features of the fluid injector system 100 and the fluid delivery set associated therewith are likewise described in detail herein. While the various examples of the methods and processes are shown with reference to an injector system having a multi-use disposable set (“MUDS”) and a single-use disposable set (“SUDS”) configuration in FIGS. 1-2, the disclosure is not limited to such an injector system and may be utilized in other syringe based injector systems, such as but not limited to those described in U.S. Pat. Nos. 7,553,294, 7,563,249, 8,945,051, 9,173,995, 10,124,110; and U.S. application Ser. Nos. 15/305,285, 15/541,573, 15/568,505; the disclosures of each of which are incorporated herein in their entirety by this reference.

With reference to FIG. 1, a fluid injector system 100 according to one example includes an injector housing 102 that encloses the various mechanical drive components, electrical and power components necessary to drive the mechanical drive components, and control components, such as electronic memory and electronic control devices, used to control operation of reciprocally movable pistons 103 (not shown) associated with the fluid injector system 100 described herein. Such pistons 103 may be reciprocally operable via electro-mechanical drive components such as a ball screw shaft driven by a motor, a voice coil actuator, a rack-and-pinion gear drive, a linear motor, and the like.

The fluid injector system 100 may include at least one bulk fluid connector 118 for connection with at least one bulk fluid source 120. In some examples, a plurality of bulk fluid connectors 118 may be provided. For example, as shown in the fluid injector embodiment illustrated in FIG. 1, three bulk fluid connectors 118 may be provided in a side-by-side or other arrangement. In some examples, the at least one bulk fluid connector 118 may include a spike configured for removably connecting to the at least one bulk fluid source 120, such as a vial, a bottle, or a bag. The at least one bulk fluid connector 118 may be formed on the multi-use disposable set (“MUDS”), as described herein. The at least one bulk fluid source 120 may be configured for receiving a medical fluid, such as saline, Ringer's lactate, an imaging contrast medium solution, or other medical fluid, for delivery to the patient by the fluid injector system 100.

With reference to FIG. 2, a MUDS 130 is configured for being removably connected to the fluid injector system 100 for delivering one or more fluids from the one or more bulk fluid sources 120 to the patient. Examples and features of embodiments of the MUDS are further described in PCT International Publication No. WO 2016/112163, filed on Jan. 7, 2016, the disclosure of which is incorporated herein by reference in its entirety. The MUDS 130 may include one or more fluid reservoirs, such as one or more syringes 132. As used herein, the term “fluid reservoir” means any container capable of taking in and delivering a fluid, for example during a fluid injection procedure including, for example a syringe, a rolling diaphragm, a pump, a compressible bag, and the like. Fluid reservoirs may include the interior volume of at least a portion of a fluid pathway, such as one or more tubing lengths, that are in fluid communication with the interior of the fluid reservoir, including fluid pathway portions that remain in fluid communication with the fluid reservoir after the system is closed or fluidly isolated from the remainder of the fluid pathway. In some examples, the number of fluid reservoirs may correspond to the number of bulk fluid sources 120 (shown in FIG. 1). For example, with reference to FIG. 2, the MUDS 130 has three syringes 132 in a side-by-side arrangement such that each syringe 132 is fluidly connectable to one or more of the corresponding three bulk fluid sources 120. In some examples, one or more bulk fluid sources 120 may be connected to one or more syringes 132 of the MUDS 130. Each syringe 132 may be fluidly connectable to one of the bulk fluid sources 120 by a corresponding bulk fluid connector 118 and an associated MUDS fluid path 134. The MUDS fluid path 134 may have a spike element that connects to the bulk fluid connector 118 and the fluid line 150. In some examples, the bulk fluid connector 118 may be provided directly on the MUDS 130.

With continued reference to FIGS. 1 and 2, the MUDS 130 may include one or more valves 136, such as stopcock valves, for controlling which medical fluid or combinations of medical fluids are withdrawn from the multi-dose bulk fluid source 120 (see FIG. 1) into the fluid reservoirs 132 and/or are delivered to a patient from each fluid reservoir 132. In some examples, the one or more valves 136 may be provided on a distal end of the plurality of syringes 132 or on a manifold 148. The manifold 148 may be in selectable fluid communication via valves 136 with the interior volume of the syringes 132. The interior volume of the syringes 132 may be in selectable fluid communication via valves 136 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the interior volume of the one or more syringes 132 or it may be delivered from the interior volume of the one or more syringes 132. In a first position, such as during the filling of the syringes 132, the one or more valves 136 are oriented such that fluid flows from the bulk fluid source 120 into the desired syringe 132 through a fluid inlet line 150, such as a MUDS fluid path. During the filling procedure, the one or more valves 136 are positioned such that fluid flow through one or more fluid outlet lines 152 or manifold 148 is blocked or closed. In a second position, such as during a fluid delivery procedure, fluid from one or more syringes 132 is delivered to the manifold 148 through the one or more fluid outlet lines 152 or syringe valve outlet ports. During the delivery procedure, the one or more valves 136 are positioned such that fluid flow through one or more fluid inlet lines 150 is blocked or closed. In a third position, the one or more valves 136 are oriented such that fluid flow through the one or more fluid inlet lines 150 and the one or more fluid outlet lines 152 or manifold 148 is blocked or closed. Thus, in the third position, each of the one or more valves 136 isolates the corresponding syringe 132 and prevents fluid flow into and out of the interior volume of the corresponding syringe 132. As such, each of the one or more syringes 132 and the corresponding valve 136 defines a closed system.

The one or more valves 136, fluid inlet lines 150, and/or fluid outlet lines 152 may be integrated into or in fluid communication via the manifold 148. The one or more valves 136 may be selectively positioned to the first or second position by manual or automatic handling. For example, the operator may position the one or more valves 136 into the desired position for filling, fluid delivery, or the closed position. In other examples, at least a portion of the fluid injector system 100 is operable for automatically positioning the one or more valves 136 into a desired position for filling, fluid delivery, or the closed position based on input by the operator or by a protocol executed by the electronic control unit.

With continued reference to FIGS. 1 and 2, according to the described embodiment the fluid injector system 100 may have a connection port 192 that is configured to form a releasable fluid connection with at least a portion of the SUDS 190. In some examples, the connection port 192 may be formed on the MUDS 130. As described herein, the SUDS 190 may be connected to the connection port 192, formed on at least a portion of the MUDS 130 and/or the housing 102. Desirably, the connection between the SUDS 190 and the connection port 192 is a releasable connection to allow the SUDS 190 to be selectively connected to and disconnected from the connection port 192. In some examples, the SUDS 190 may be disconnected from the connection port 192 and disposed after each fluid delivery procedure, and a new SUDS 190 may be connected to the connection port 192 for a subsequent fluid delivery procedure. The SUDS 190 may be used to deliver one or more medical fluids to a patient by SUDS fluid line 208 having a distal end that may be selectively disconnected from the body of the SUDS 190 and connected to a patient catheter. Other examples and features of the SUDS 190 are described in U.S. Patent Publication No. 2016/0331951, filed Jul. 7, 2016, the disclosure of which is incorporated herein by reference.

Referring again to FIG. 1, the fluid injector system 100 may include one or more user interfaces 124, such as a graphical user interface (GUI) display window. The user interface 124 may display information pertinent to a fluid injection procedure involving fluid injector system 100, such as injection status or progress, current flow rate, fluid pressure, and volume remaining in the at least one bulk fluid source 120 connected to the fluid injector system 100 and may be a touch screen GUI that allows an operator to input commands and/or data for operation of fluid injector system 100. Additionally, the fluid injector system 100 and/or user interface 124 may include at least one control button 126 for tactile operation by an attendant operator of the fluid injector system 100. The at least one control button 126 may be a graphical part of the user interface 124, such as a touch screen.

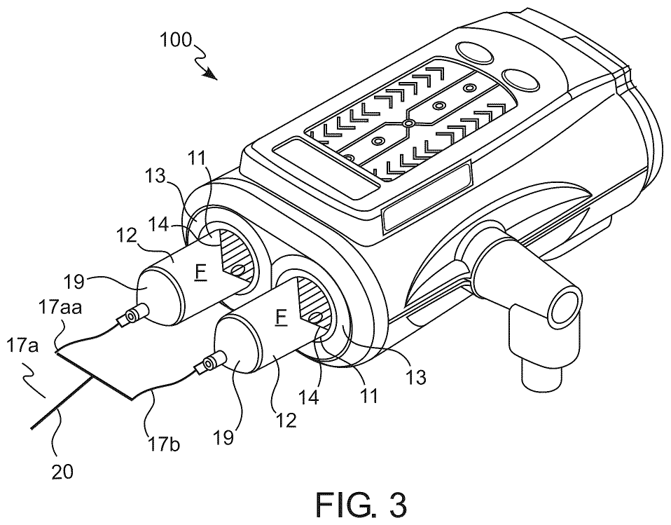

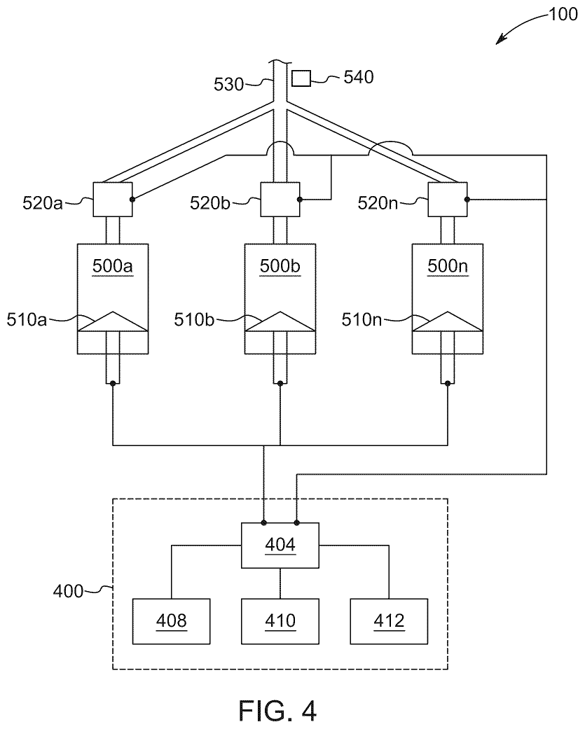

While FIGS. 1-2 illustrate one example of a fluid injector system 100 and associated components and structure, it is to be understood that the present disclosure is not limited to any particular type or variety of the fluid injector system 100. Referring now to FIG. 3, another non-limiting example of a fluid injector system 100 in accordance with the present disclosure includes at least one fluid reservoir, such as syringe 12, at least one piston 103 (see FIG. 4) connectable to at least one plunger 14, and a fluid control module (not pictured). The at least one syringe 12 is generally adapted to interface with at least one component of the system, such as a syringe port 13. The fluid injector system 100 is generally configured to deliver at least one fluid F to a patient during an injection procedure. The fluid injector system 100 is configured to releasably receive the at least one syringe 12, which is to be filled with at least one fluid F, such as a contrast media, saline solution, Ringer's lactate, or any desired medical fluid. The system may be a multi-syringe injector, wherein several syringes may be oriented side-by-side or in another spatial relationship and are separately actuated by respective pistons associated with the injector. The at least one syringe 12 may be oriented in any manner such as upright, downright, or positioned at any degree angle. In another embodiment, a fluid injector 100 may interface with one or more rolling diaphragm syringes (not shown). Non-limiting examples of rolling diaphragm syringe based injectors are described in U.S. application Ser. Nos. 15/305,285, and 15/568,505 and PCT International Application No. PCT/US2017/056747, the disclosures of which are incorporated herein.

With continued reference to FIG. 3, the injector system 100 may be used during a medical procedure to inject the at least one medical fluid F into the vasculature system of a patient by driving a plunger 14 of at least one syringe 12 with a drive member, such as the at least one piston 103 (see FIG. 4). The at least one piston may be reciprocally operable upon at least a portion of the at least one syringe, such as the plunger 14. Upon engagement, the at least one piston may move the plunger 14 toward the distal end 19 of the at least one syringe, as well as retracting the plunger 14 toward the proximal end 11 of the at least one syringe 12.

A tubing set 17 (e.g., first and second fluid conduits 17a and 17b, and common fluid conduit 20) may be in fluid communication with an outlet port of each syringe 12 to place each syringe in fluid communication with a catheter for delivering the fluid F from each syringes 12 to the catheter (not shown) inserted into a patient at a vascular access site. The first and second fluid conduits 17a and 17b may be connected to the common fluid conduit 20 by any suitable mechanism known in the art (e.g., a Y-connector or a T-connector). The fluid injector system 100 shown in FIG. 3 is an open system do to the lack of valves capable of isolating the syringes 12 from one another and from at least a portion of the tubing set 17. However, it is to be understood that valves, similar or identical to the valves 136 described with reference to the fluid injector system 100 of FIGS. 1 and 2, may be added distally of the syringes 12 to convert fluid injector system 100 of FIG. 3 to a closed system.

Referring now to FIG. 4, fluid injector systems 100 in accordance with the present disclosure may be associated with and controlled by an electronic control device 400 configured to execute one or more injector protocols including, for example, the filling, priming, and delivery operations. In some examples, the electronic control device 400 may control the operation of various valves, stopcocks, piston members, and other elements to affect a desired gas/air removal, filling, and/or delivery procedure. The electronic control device 400 may include at least one processor 404, memory 408, an input component 410, and an output component 412. The electronic control device further may include a bus that permits communication among the components of electronic control device 400. The at least one processor 404 may be implemented in hardware, firmware, or a combination of hardware and software. For example, processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 408 may include a hard disk (e.g., a magnetic disk, an optical disk, a magneto-optic disk, a solid state disk, etc.) and/or another type of computer-readable medium. The input component 410 may include a component that permits the electronic control device 400 to receive information, such as via user input (e.g., the user interface 124). The output component 412 may include a component that provides output information from the electronic control device 400 (e.g., the user interface 124).

The electronic control device 400 may be programmed or configured to perform one or more processes and/or methods based on the at least one processor 404 executing software instructions stored by a computer-readable medium, such as memory 408. When executed, software instructions stored in memory 408 may cause the at least one processor 404 to perform one or more processes and/or methods described herein.

With continued reference to FIG. 4, the electronic control device 400, more particularly the at least one processor 404, may be in operative communication with one or more components of the fluid injector system 100 to control an operation of the fluid injector system 100. The electronic control device 400 may be in operative communication with one or more drive components 510a, 510b, 510n respectively associated with one or more fluid reservoirs 500a, 500b, 500n of the fluid injector system 100 to control filling of fluid and delivery of fluid from the fluid reservoirs 500a, 500b, 500n. More particularly, each of the one or more drive components 510a, 510b, 510n may be associated with one of the fluid reservoirs 500a, 500b, 500n such that fluid contained in each of the fluid reservoirs 500a, 500b, 500n may be selectively delivered via actuation of the associated drive component 510a, 510b, 510n. The fluid reservoirs 500a, 500b, 500n may be, or may correspond to, the syringes 132 of the fluid injector system 100 of FIGS. 1-2 and/or the syringes 12 of the fluid injector system 100 of FIG. 3 or other syringe-type structures, such as rolling diaphragm syringes, as described herein. The one or more drive components 510a, 510b, 510n may be, or may correspond to, the pistons 103 of the fluid injector systems 100 of FIGS. 1-3. The one or more fluid reservoirs 500a, 500b, 500n may be in fluid communication with a fluid conduit 530 for delivering fluid to a catheter or other component connected to a patient. The fluid conduit 530 may be, or may correspond to, the SUDS 190 of the fluid injector system 100 of FIGS. 1-2 and/or the tubing set 17 of the fluid injector system 100 of FIG. 3

In aspects and examples of a closed fluid injector system 100 (e.g., the fluid injector system 100 of FIGS. 1 and 2), the electronic control device 400 further may be in operative communication with one or more valves 520a, 520b, 520n in order to rotate or otherwise actuate the valves 520a, 520b, 520n to direct flow into or out of and/or isolate flow from one or more of the fluid reservoirs 500a, 500b, 500n to the fluid conduit 530. The valves 520a, 520b, 520n may be, or may correspond to, the valves 136 described herein in connection with FIG. 2.

In some aspects and examples, the at least one processor 404 may be programmed or configured to execute an injection protocol including a first phase and at least a second phase. Each of the first phase and the second phase of the injection protocol include or define a desired steady-state ratio of a first fluid relative to a second fluid. According to various embodiments, the steady-state ratio for each phase may range from 100:0 of the first fluid to the second fluid to 0:100 of the first fluid to the second fluid, inclusive of any intermediate ratios. As described herein, the desired steady-state ratio is based on volume component of the first fluid relative to the volume component of the second fluid, although mass, density, viscosity, flow rate, or another characteristic of the fluids may also be the basis of the desired steady-state ratio. In various embodiments, the first fluid may be an imaging contrast solution and the second fluid may be a flushing fluid, such as saline or Ringer's Lactate.

In some aspects or examples, the first fluid may be contained in a first fluid reservoir 500a and the second fluid may be contained in a second fluid reservoir 500b. It should be understood that the order of the various fluid reservoirs may be changed, for example, in certain embodiments the first fluid reservoir may be reservoir 500b and the second fluid reservoir may be 500a without deviating from the scope of the present disclosure. In certain embodiments, the third fluid reservoir 500n may contain a third fluid or an additional volume of the first or second fluids, or a different concentration of the first or second fluid. The desired steady-state ratios for the first and second phases of the injection protocol may be reached by selectively and independently actuating the first and second drive components 510a, 510b associated with the first and second fluid reservoir 500a, 500b and optionally actuating a third drive component 510n associated with a third fluid reservoir 500n. For example, if the desired steady-state ratio is 50% of the first fluid to 50% of the second fluid, the first and second drive components 510a, 510b associated with the first and second fluid reservoir 500a, 500b may be actuated, by the at least one processor 404, at the same speed to facilitate equal delivery of the first and second fluids. Similarly, if the desired steady-state ratio is 75% of the first fluid to 25% of the second fluid, the first drive component 510a associated with the first fluid reservoir 500a may be actuated at three times the speed of the second drive component 510b associated with the second fluid reservoir 500b to facilitate delivery of a 3:1 ratio of the first fluid relative to the second fluid. If the desired steady-state ratio is 100% of the first fluid to 0% of the second fluid, the first drive component 510a associated with the first fluid reservoir 500a is actuated and the second drive component 510b associated with the second fluid reservoir 500b is not actuated. In aspects or examples of the present disclosure, the first fluid may be contrast or another diagnostic imaging fluid, and the second fluid may be a diluent such as saline, Ringer's lactate, a mixture of contrast diluted with saline, or the like. The terms “contrast” and “diluent” may be used herein to refer to the first fluid and second fluid, respectively, when describing specific aspects or examples of the present disclosure. However, it is to be understood that embodiments of the present disclosure are not limited to using contrast and diluent as the first and second fluids.

However, when the controller instructs the various drive components to drive as a specific rate to achieve a desired steady-state fluid ratio, the observed initial fluid ratio may differ from the desired steady-state ratio due to one or more fluid flow factors (e.g., fluid ratio of the previous phase, differences in the densities and/or viscosities of the first and at least the second fluids, impedance and/or capacitance of the one or more fluid reservoirs 500a, 500b, 500n and the fluid conduit 530, and/or fluid dynamics within the fluid reservoirs 500a, 500b, 500n and the fluid conduit 530), and the actual ratio of the first and second fluid delivered to the patient may not be reflective of the desired steady-state ratio. Aspects or examples of the present disclosure are directed to fluid injector systems, computer program products, and methods of compensating for these fluid flow factors so that the initially observed fluid ratio more quickly and closely matches the desired fluid ratio.

In some aspects of examples, the ratio of the first fluid relative to the second fluid may be measured by a ratio sensor 540 associated with the fluid conduit 530 downstream of the first and second fluid reservoirs 500a, 500b. The ratio sensor 540 may be operatively connected to the at least one processor 404. In this manner, the first and second drive components 510a, 510b associated with the first and second fluid reservoir 500a, 500b may be actuated, by the at least one processor 404, at a desired speed to achieve a desired ratio of the first and second fluids, as sensed by the ratio sensor 540. Based on a sensed density, the at least one processor 404 may increase or decrease the speed of the first and second drive components 510a, 510b such that the actual fluid ratio sensed by the ratio sensor 540 is substantially equal to a desired ratio. In some aspects of examples, the ratio sensor 540 may be a density sensor configured for sensing a density of the fluid(s) flowing through the fluid conduit 530. In other aspects of examples, the ratio sensor 540 may be a viscosity sensor configured for sensing a viscosity of the fluid(s) flowing through the fluid conduit 530. In some aspects of examples, the ratio sensor 540 may be a light refraction sensor configured for sensing a ratio of the fluid(s) flowing through the fluid conduit 530 based on light refraction properties of the fluid(s). In further aspects or examples, the ratio sensors 540 may be a plurality of ratio sensors 540, including any combination of one or more of density sensors, viscosity sensors, and light refraction sensors.

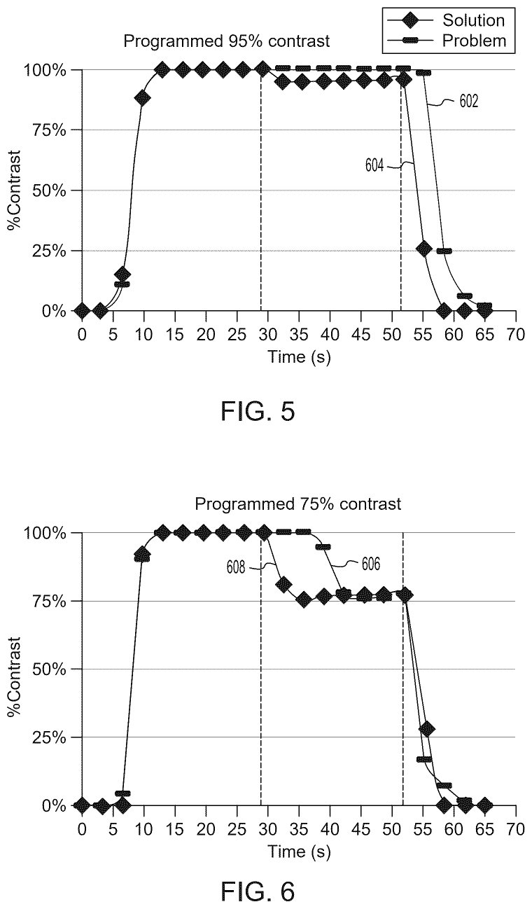

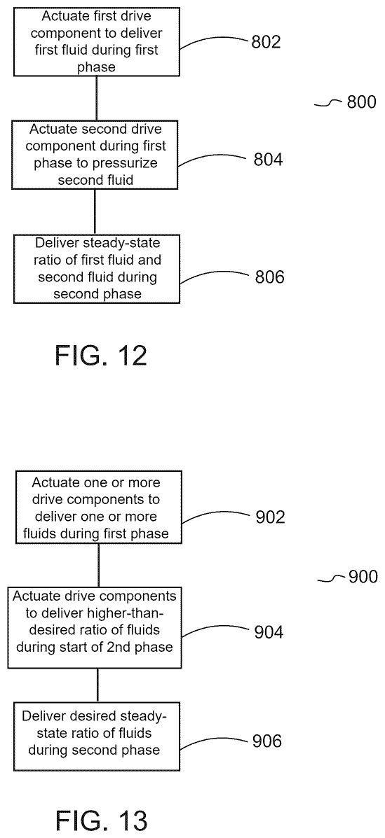

FIGS. 5-8 illustrate flow ratio curves according to aspects or examples of the present disclosure, which account for the above fluid flow factors, compared to flow ratio curves of prior art fluid injector systems. Referring now to FIG. 5, flow ratio curves are shown for an injection protocol including a first phase having a desired steady-state ratio of 100% of the first fluid, such as contrast to 0% of the second fluid, such as a saline diluent, and a second phase having a desired steady-state ratio of 95% of the first fluid to 5% of the second fluid. The first phase injection occurs from approximately 0 seconds to approximately 29 seconds, while the second phase occurs from approximately 29 seconds into the injection to approximately 52 seconds. Flow ratio curve 602 illustrates the actual observed ratio of first fluid to the second delivered to the patient during the operation of certain prior art fluid injector systems and protocols. Flow ratio curve 604 illustrates the actual ratio of contrast to diluent delivered to the patient during the operation of the fluid injector system 100 according to various protocols of the present disclosure. As can be appreciated from FIG. 5, the flow ratio curve 602 never reaches the desired steady-state ratio of 95% contrast to 5% diluent due to one or more of the fluid flow factors described herein. In contrast, the flow ratio curve 604 reaches the desired steady-state ratio of 95% contrast to 5% diluent at approximately 32 seconds by performing one or more of the processes and/or methods described herein (see FIGS. 12 and 13).

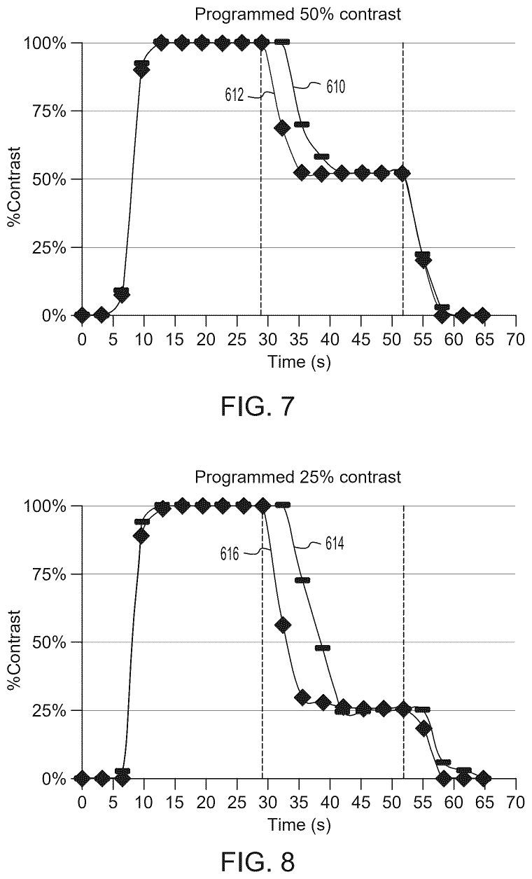

Similar to FIG. 5, FIGS. 6-8 illustrate flow ratio curves for various injection protocol having the first phase occurring from approximately 0 seconds to approximately 29 seconds, and the second phase occurring from approximately 29 seconds to approximately 52 seconds. In FIG. 6-8, flow ratio curves 606, 610, 614 illustrate the actual ratio of the first fluid (e.g., contrast) to the second fluid (e.g., diluent) that is delivered to the patient during the operation of certain prior art fluid injector systems and protocols, while flow ratio curves 608, 612, 616 illustrate the actual ratio of the first fluid (e.g., contrast) to the second fluid (e.g., diluent) delivered to the patient during the operation of the fluid injector system 100 according to the present disclosure. The first phase of the injection protocols of each of FIGS. 6-8 has a desired steady-state ratio of 100% contrast to 0% diluent. The second phase of the injection protocol of FIG. 6 has a desired steady-state ratio of 75% contrast to 25% diluent; the second phase of the injection protocol of FIG. 7 has a desired steady-state ratio of 50% contrast to 50% diluent; and the second phase of the injection protocol of FIG. 8 has a desired steady-state ratio of 25% contrast to 75% diluent. As may be appreciated from FIGS. 6-8, the flow ratio curves 606, 610, 614 corresponding to the prior art fluid injector systems take longer to reach the desired steady-state ratio of the second phase as compared to the flow ratio curves 608, 612, 616 corresponding to the fluid injector system 100 of the present disclosure. While the examples illustrated in FIGS. 5-8 illustrate injection protocols where the first phase is 100% contrast to 0% diluent, other ratios of the first fluid to the second fluid may be used in the first phase of the injection protocols and similar delays in the second phase reaching the desired steady-state flow ratios are also observed using conventional injection protocols, whereas using protocols that account for the one or more fluid flow factors as described herein reach the steady-state fluid flow ratio in significantly reduced times. Similar reductions in times to reach steady-state for a third fluid phase are also observed using the various protocols that account for the one or more fluid flow factors as described herein are also observed.

The reduction in time to reach the desired steady-state pressure ratio of the second phase of flow ratio curves 608, 612, 616 ensures that the volumetric ratio of the first fluid relative to the second fluid actually delivered to the patient is substantially consistent with the programmed injection protocol. Referring now to FIGS. 12 and 13, methods 800 and 900 for performing the injection protocol to decrease the time required to reach the desired steady-state ratio of the second phase will now be described. In some aspects or examples, each of the methods 800 and 900 may be performed by the at least one processor 404 of the fluid injector system 100 as described herein with reference to FIGS. 1-4. In some aspects or examples, each of the methods 800 and 900 may be stored as instructions in a non-transitory computer-readable media, such as memory 408 of FIG. 4, such that the instructions, when executed by the at least one processor 404, cause the at least one processor 404 to perform the method 800 and/or the method 900.

First referring to FIG. 12, at step 802 the method 800 may include, during the first phase of the injection protocol, actuating at least the first drive component 510a to inject the fluid prescribed in the first phase. To provide clarity of the present disclosure and for simplicity, the following methods describe an injection protocol where the first phase is 100% of the first fluid and 0% of the second fluid, Actuation of the first drive component 510a pressurizes the first fluid reservoir 500a and injects the first fluid into the fluid conduit 530 and ultimately to the patient. In some aspects or examples, the first phase of the injection protocol is a dual flow phase including both the first fluid and the second fluid and includes the second fluid in addition to the first fluid, in which case the second drive component 510b is actuated to pressurize and inject the second fluid from the second fluid reservoir 500b. Similarly, additional fluids prescribed in the first phase of the injection protocol may be pressurized and injected from the additional fluid reservoirs 500n by actuating the associated additional drive components 510n. Actuating any of the drive components 510a, 510b, 510n may include distally advancing the drive component 510a, 510b, 510n to reduce the internal volume of the associated fluid reservoir 500a, 500b, 500n. Drive components 510a, 510b, 510n may be advanced at a constant speed, at a linearly increasing or decreasing speed, at an exponentially increasing or decreasing speed, or any other speed profile. Actuation of the one or more drive components 510a, 510b, 510n may be controlled by the at least one processor 440.

In aspects or examples in which the fluid injector system 100 is a closed system, the method 800 further may include closing the second valve 520b prior to step 802 to prevent flow from the fluid conduit 530 into the second fluid reservoir 500b.

With continued reference to FIG. 12, the method 800 further may include, at step 804, during the first phase of the injection protocol prior to transitioning to the second phase of the injection protocol, actuating the second drive component 510b to pressurize the second fluid in the second fluid reservoir 500b relative to a pressure of the first fluid injected into in the fluid conduit 530 at step 802. In aspects or examples in which the fluid injector system 100 is a closed system, the method 800 further may include opening the second valve 520b prior to step 804 to allow fluid communication between the second fluid reservoir 500b and the fluid conduit 530.

In aspects or examples in which the first phase includes the injection of multiple fluids into the fluid conduit 530 at step 802, the second fluid is pressurized at step 804 relative to a total pressure of all the fluid within the fluid conduit 530. Pressurizing the second fluid in the second fluid reservoir 500b may include advancing the second drive component 510b to oppose the pressure of the fluid in the fluid conduit 530 and thereby prevent backflow of the fluid in the fluid conduit 530 towards the second fluid reservoir 500b. The desired steady-state ratio of the first fluid and the second fluid in the second phase of the injection protocol is reached at a quicker rate than if the second drive component 510b was not actuated to pressurize the second fluid reservoir 500b prior to transitioning to the second phase of the injection protocol (see, e.g., FIGS. 5-8). Actuating the second drive component 510b to pressurize the second fluid reservoir 500b may be controlled by the at least one processor 440.

In some aspects or examples, pressurizing the second fluid relative to the pressure of the first fluid includes selecting a pressure ratio of the pressure of the first fluid and a pressure of the second fluid to reach the desired steady-state flow ratio of the first fluid and the second fluid for the second phase of the injection protocol. In some aspects or examples, pressurizing the second fluid relative to the pressure of the first fluid includes gradually increasing the pressure of the second fluid such that at the transition from the first phase of the injection protocol to the second phase of the injection protocol, the pressure of the second fluid in the second fluid reservoir 500b is substantially equal to the pressure of the first fluid in the fluid conduit 530. In other aspects or examples, pressurizing the second fluid relative to the pressure of the first fluid may include gradually increasing a pressure of the second fluid such that, at the transition from the first phase of the injection protocol to the second phase of the injection protocol, the pressure of the second fluid in the second fluid reservoir 500b is 20% to 150% of the pressure of the first fluid in the fluid conduit 530. In addition to obtaining more accurate fluid flow profiles, the present disclosure may also prevent backflow from the higher pressure syringe into the lower pressure syringe, which can occur where there is a difference in pressure between the syringes (see, PCT International Application No. PCT/US2019/048249, filed Aug. 27, 2019 entitled “Fluid Injector System, Method of Preventing Fluid Backflow, and Computer Program Product”, the disclosure of which is incorporated herein by reference.

With continued reference to FIG. 12, the method 800 further may include, at step 806, during the second phase of the injection protocol, actuating the second of the drive component 510b to inject the second fluid from the second fluid reservoir 500b through the fluid conduit 530 so that the desired steady-state ratio of the first fluid and the second fluid in the second phase is reached. In some aspects or examples, the second phase of the injection protocol includes the first fluid in addition to the second fluid, in which case the first drive component 510a is actuated, or remains actuated following the first phase, to pressurize and inject the first fluid from the first fluid reservoir 500a. In some aspects or examples, step 806 further includes adjusting a speed of the first drive component 510a to inject the first fluid at a desired flow rate to reach the desired steady-state ratio of the first fluid and the second fluid through the fluid conduit 530 during the second phase of the injection protocol. That is, the speed of the first drive component 510a may be adjusted to transition from the desired steady-state ratio of the first phase of the injection protocol to the desired steady-state ratio of the second phase of the injection protocol.

It will be understood by one of skill in the art that similar protocols and methods may include a third or more fluids that may be included in the fluid flow to reach steady-state without deviating from the scope of the present disclosure. Additional fluids prescribed in the second phase of the injection protocol may be pressurized and injected from the additional fluid reservoirs 500n by actuating the associated additional drive components 510n. Actuating any of the drive components 510a, 510b, 510n may include distally advancing the drive component 510a, 510b, 510n to reduce the internal volume of the associated fluid reservoir 500a, 500b, 500n. The drive components 510a, 510b, 510n may be advanced at a constant speed, at a linearly increasing or decreasing speed, at an exponentially increasing or decreasing speed, or any other speed profile, including, for example a pulsed speed profile. Actuation of the drive components 510a, 510b, 510n may be controlled by the at least one processor 440.

In aspects or examples in which the fluid injector system 100 is a closed system, the method 800 further may include closing the second valves 520b prior pressurizing the second fluid reservoir 500b at step 804 to prevent flow from the fluid conduit 530 into the second fluid reservoir 500b. Further, closing of the second valve 520b prior to pressurizing the second fluid reservoir 500b at step 804 may also reduce the volume and pressure effects due to compliance volume of the second fluid reservoir 500b and system slack associated with the second drive component 510b. In some aspects or examples, the second valve 520b may be closed prior to injecting the first phase of the injection protocol at step 802. With the second valve 520b closed, the second fluid reservoir 500b is isolated from the fluid conduit 530 and the other fluid reservoirs 500a, 500n. As such, backflow of the first fluid into the second fluid reservoir 500b is prevented. During the transition to the second phase of the injection protocol at step 806, the second valve 520b may be at least partially opened to allow the second fluid from the second fluid reservoir 500b to be injected into the fluid conduit 530. When the second valve 520b is opened, the pressure generated in the second fluid reservoir 500b at step 804 may overcome the pressure of the fluid previously injected into the fluid conduit 530 (at step 802), allowing the second fluid to advance into the fluid conduit 530 and mix with the first fluid without having to overcome the pressure differential of the pressurized first fluid and the compliance effects from pressurizing the second fluid reservoir 500b, thereby reducing the time required to reach the desired steady-state ratio and desired flow profile for the first fluid and the second fluid of the second phase of the injection protocol.

Referring now to FIG. 13, another method for reducing the time required to reach the desired steady-state ratio of the second phase of the injection protocol is shown. At step 902 the method 900 may include, during the first phase of the injection protocol, actuating one or more of the drive components 510a, 510b, 510n to inject at least one fluid from the one or more fluid reservoirs 500a, 500b, 500n through the fluid conduit 530 at the desired steady-state ratio for the first phase of the injection protocol. Actuating any of the drive components 510a, 510b, 510n may include distally advancing the drive component 510a, 510b, 510n to reduce the internal volume of the associated fluid reservoir 500a, 500b, 500n. The drive components 510a, 510b, 510n may be advanced at a constant speed, at a linearly increasing or decreasing speed, at an exponentially increasing or decreasing speed, or any other speed profile. Actuation of the drive components 510a, 510b, 510n may be controlled by the at least one processor 440.

In aspects or examples in which the fluid injector system 100 is a closed system, the method 900 further may include closing the second valve 520b prior to step 902 to prevent flow from the fluid conduit 530 into the second fluid reservoir 500b.

With continued reference to FIG. 13, the method 900 further may include, at step 904, during an initial portion of the second phase of the injection protocol, actuating the first and second drive component 510a, 510b to inject the first fluid and the second fluid through the fluid conduit 530 such that a ratio of volume of the second fluid displaced from the second fluid reservoir 500b relative to volume of the first fluid displaced from the first fluid reservoir 500a exceeds the desired steady-state ratio (for example as a modified steady-state volumetric ratio) and desired flow profile for the first fluid and the second fluid for the second phase. For example, is the desired steady-state ratio for the second phase is includes 10% of the second fluid to 90% of the first fluid, the first and second drive component 510a, 510b may be actuated by the at least one processor 440 to generate a ratio of 20% of the second fluid to 80% of the first fluid. The increase in the ratio of the second fluid relative to the first fluid assists the second fluid in overcoming the pressure of the first fluid flowing through the fluid conduit 530, thereby further decreasing the time required to reach the desired steady-state ratio and desired flow profile for the first fluid and the second fluid of the second phase of the injection protocol. Thus, the desired steady-state ratio of the first fluid and the second fluid in the second phase is reached more quickly than if the first and second drive component 510a, 510b were actuated such that a ratio of volume of the second fluid displaced from the second fluid reservoir 500b relative to volume of the first fluid displaced from the first fluid reservoir 500a did not exceed the desired steady-state ratio of the second phase. In certain embodiments, the modified steady-state volumetric ratio of the minor fluid of the dual flow system may range from 1.1 times the desired ratio component for the minor fluid up to 5.0 times the desired ratio component for the minor fluid.

In aspects or examples in which the fluid injector system 100 is a closed system, the method 900 further may include opening the second valve 520b prior to step 904 to allow flow from the second fluid reservoir 500b into the fluid conduit 530.

With continued reference to FIG. 13, the method 900 further may include, at step 906, subsequent to the initial portion of the second phase, actuating the first and second drive component 510a, 510b to reduce the volumetric ratio of the second fluid relative to the first fluid injected at step 904 until the volumetric ratio reaches the second desired steady-state ratio and desired flow profile for the first fluid and the second fluid in the second phase. That is, after the initial injection of the first fluid and the second fluid at the modified volumetric ratio of step 804, the ratio of the first fluid and the second fluid is adjusted to the desired steady-state ratio of the second phase. In certain embodiments, the adjustment to the desired steady-state ratio may be performed gradually over time to smoothly adjust the ratio of fluid delivered to the patient towards the desired steady-state ratio of the second phase. According to other embodiments, the adjustment to the desired steady-state ratio may be quickly adjusted after a set period of time. Appropriate modified steady-state volumetric ratios for a desired steady-state ratio of a specific phase or a specific injection protocol may be stored in a memory associated with the controller, such as in a “look-up table”, or may be determined by one or more algorithmic calculation.

In some aspects or examples, the method 900 further may include, during the first phase of the injection protocol prior to transitioning to the second phase of the injection protocol, actuating the second drive component 510b to pressurize the second fluid in the second fluid reservoir 500b relative to a pressure of the first fluid injected into the fluid conduit 530. Pressurizing the second fluid may be performed in same manner as described herein with reference to step 804 of the method 800. In aspects or examples in which the fluid injector system 100 is a closed system, the method 900 further may include closing the second valve 520b prior to pressurizing the second fluid reservoir 500b. With the second valve 520b closed, the second fluid reservoir 500b is isolated from the fluid conduit 530 and the other fluid reservoirs 500a, 500n. As such, backflow into the second fluid reservoir 500b is prohibited. During the transition to the second phase of the injection protocol at step 906, the second valve 520b may be opened to allow the second fluid from the second fluid reservoir 500b to be injected into the fluid conduit 530. When the second valve 520b is opened, the pressure generated in the second fluid reservoir 500b may overcome the pressure of the fluid previously injected into the fluid conduit 530 (at step 902), allowing the second fluid to advance into the fluid conduit 530 and reducing the time required to reach the desired steady-state ratio of the second phase of the injection protocol.

In some aspects or examples, the methods 800, 900 further may include, during the first phase of the injection protocol prior to transitioning to the second phase of the injection protocol, ceasing actuation or reducing the speed of the first drive component 510a to reduce the pressure of the first fluid in the fluid conduit 530. Due to the cessation of speed reduction of the first drive component 510a, the fluid pressure within the fluid conduit 530 is temporarily reduced at the start of the second phase of the injection protocol. Consequently, the second fluid injected into the fluid conduit 530 at the beginning of the second phase must only overcome the reduced pressure of the first fluid in the fluid conduit 530, allowing the desired steady-state ratio of the second phase of the injection protocol to be reached more quickly than if the first drive component 510a was not reduced in speed. Once the second fluid has begun flowing into the fluid conduit 530, the speed of the first drive component 510a may be returned to the speed prescribed by the injection protocol to achieve the desired steady-state ratio of the second phase.

In some aspects or examples, the methods 800, 900 further may include, during the first phase of the injection protocol prior to transitioning to the second phase of the injection protocol, adjusting at least one property of the injection protocol to prevent backflow of the fluid from the fluid conduit 530 towards the second fluid reservoir 500b. In some aspects or examples, adjusting at least one property of the injection protocol may include pulsing the second drive component 510b to generate a pressure front within the fluid conduit 530 that prevents the fluid in the fluid conduit 530 from flowing toward the second fluid reservoir 500b. Exemplary processes and methods for pulsing the second drive component 510b are described in PCT International Application No. PCT/US2019/048249.

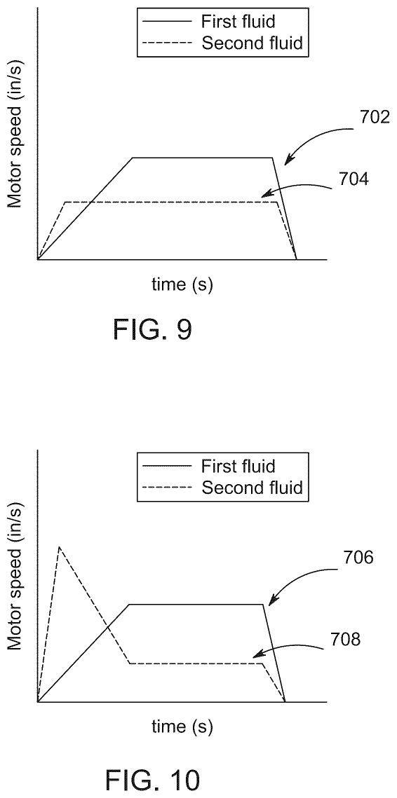

In some aspects or examples, adjusting at least one property of the injection protocol may include advancing one or more of the drive components 510a, 510b, 510n at a constant speed, linearly increasing the speed of one or more of the drive components 510a, 510b, 510n, and/or exponentially increasing the speed of one or more of the drive components 510a, 510b, 510n. FIGS. 9-11 illustrate various motor speed profiles for the first and second drive component 510a, 510b during the second phase of the injection protocol. Referring first to FIG. 9, a motor speed profile 702 for the first drive component 510a is compared to a motor speed profile 704 for the second drive component 510b. As can be appreciated from FIG. 9, both of the motor speed profiles 702, 704 gradually increase from zero to a constant speed, with the constant speed corresponding to motor speed at the desired steady-state ratio of the first fluid and the second fluid. However, the motor speed profile 704 of the second fluid increases toward its constant speed at a faster rate relative to the motor speed profile 702. The result of actuating the first and second drive component 510a, 510b in accordance with the motor speed profiles 702, 704, respectively, is that the second fluid is initially delivered at a faster rate and in greater volume, thereby overcoming the pressure of the first fluid in the fluid conduit 530. As such, the desired steady-state ratio of the first fluid and the second fluid is reached more quickly than if the motor speed profiles 702, 704 increased towards their respective constant speeds at the same rate.

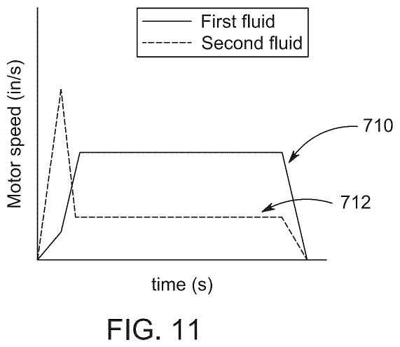

Similar to FIG. 9, FIGS. 10 and 11 illustrate comparisons of motor speed profiles 706, 710 for the first drive component 510a relative to motor speed profiles 708, 712 for the second drive component 510b. In both FIGS. 10 and 11, motor speed profiles 708, 712 for the second drive component 510b initially increase at a faster rate than motor speed profiles for the first drive component 510a. Additionally, motor speed profiles 708, 712 each include an initial spike during which the motor speed increases to a maximum speed above the constant speed, and the speed is subsequently decreased to the constant speed. The spikes in motor speed profiles 708, 712 enhance the effect of overcoming the pressure of the first fluid in the fluid conduit 530 to more quickly reach the desired steady-state ratio of the first fluid and the second fluid.

In some aspects and examples of a closed fluid injector system 100 (e.g., the fluid injector system 100 of FIGS. 1 and 2), the at least one processor 404 may be programmed or configured to execute an injection protocol including a single phase that is a dual flow phase including at least a first fluid delivered to the fluid conduit 530 from the first fluid reservoir 500a via actuation of the first drive component 510a and at least a second fluid delivered to the fluid conduit 530 from the second fluid reservoir 500b via actuation of the second drive component 510b. Such single phase, multi-fluid injection protocol may include or define a desired steady-state ratio of a first fluid relative to at least a second fluid. According to various embodiments, the steady-state ratio of fluids in the single phase may range from 99:1 of the first fluid to the second fluid to 1:99 of the first fluid to the second fluid, inclusive of any intermediate ratios.

The desired steady-state ratio for the first and second fluid of the injection protocol may be reached by selectively and independently actuating the first and second drive components 510a, 510b and the first and second valves 520a, 520b associated with the first and second fluid reservoir 500a, 500b and optionally actuating a third drive component 510n and a third valve 520n associated with a third fluid reservoir 500n. In order to compensate for effects of one or more fluid flow factors (e.g., fluid ratio of the two or more fluids, differences in the densities and/or viscosities of the two or more fluids, system slack, impedance and/or capacitance of the two or more fluid reservoirs) affecting the desired fluid ratio, the controller 404 instructs the various drive components and valves to drive at a specific rate and open at a specific time such that the volumetric ratio of the first fluid relative to the second fluid actually delivered to the patient is substantially consistent with the programmed injection protocol.

In aspects or examples in which the fluid injector system 100 is a closed system, the first and second valves 510a, 520b are initially closed. The controller 404 may instruct the first drive component 510a to pressurize the first fluid in the first fluid reservoir 500a to a first pressure and a instruct the second drive component 510b to pressurize the second fluid in the second fluid reservoir 500b to a second pressure. During pressurizing of the first and second fluid reservoirs 500a, 500b, the first and second fluids are isolated from the fluid conduit 530 due to the closed state of the first and second valves 520a, 520b. Depending on the fluid flow factors, the first pressure may be lower, substantially equal to, or higher than the second pressure. In some aspects or examples, pressurizing the second fluid relative to the pressure of the first fluid includes selecting a pressure ratio of the pressure of the first fluid and a pressure of the second fluid to reach the desired initial flow ratio that matches the desired steady-state flow ratio of the first fluid and the second fluid.

Upon reaching the desired first and second pressure, the first and second valves 520a, 520b are opened and actuation of the first and second drive components 510a, 510b is continued, thereby allowing the pressurized first and second fluids to be delivered from the first and second reservoirs 500a, 500b to the fluid conduit 530 through the first and second valves 520a 520b. The first and second drive components 510a, 510b may be advanced at a constant speed, at a linearly increasing or decreasing speed, at an exponentially increasing or decreasing speed, or any other speed profile. Actuation speed of the first and second drive components 510a, 510b prior to opening the first and second valves 520a, 520b may increase, decrease, or remain the same as the actuation speed after opening of the first and second valves 520a, 520b. The desired steady-state ratio of the first fluid and the second fluid is reached at a quicker rate than if the first and second drive components 510a, 510b were not actuated to pressurize the first and second fluid reservoirs 500a 500b.

It will be understood by one of skill in the art that similar protocols and methods may include a third or more fluids that may be included in the fluid flow to reach steady-state without deviating from the scope of the present disclosure. Additional fluids prescribed in the second phase of the injection protocol may be pressurized and injected from the additional fluid reservoirs 500n by actuating the associated additional drive components 510n and additional valves 520n. As described herein, actuation of the drive components 510a, 510b, 510n and valves 520a, 520b, 520n may be controlled by the at least one processor 440.

While examples of fluid injector systems, methods of operation thereof, and computer program products were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

We claim:

1. A computer program product for performing an injection protocol using a fluid injector system, the injection protocol including at least a first phase and a second phase subsequent to the first phase, the computer program product comprising:
non-transitory computer readable media comprising one or more instructions that, when executed by at least one processor of the fluid injector system, cause the at least one processor to:
during the first phase of the injection protocol, actuate at least a first drive component of the fluid injector system to inject the first phase including a first fluid through a fluid conduit;
during the first phase of the injection protocol prior to transitioning to the second phase of the injection protocol, actuate a second drive component of the fluid injector system to pressurize a second fluid relative to a pressure of a first fluid in the fluid conduit; and
during the second phase of the injection protocol, actuate the second drive component to inject at least the second fluid through the fluid conduit so that a desired steady-state ratio of the first fluid and the second fluid in the second phase is reached.

2. The computer program product of claim 1, wherein the first fluid comprises a contrast medium and the second fluid comprises a diluent.

3. The computer program product of claim 1, wherein the desired steady-state ratio of the first fluid and the second fluid in the second phase is reached at a quicker rate than if the second drive component is not actuated prior to transitioning to the second phase of the injection protocol.

4. The computer program product of claim 1, wherein the one or more instructions, when executed by the at least one processor of the fluid injector system, further cause the at least one processor to: during the first phase of the injection protocol prior to transitioning to the second phase of the injection protocol, cease actuation or reduce a speed of the first drive component.

5. The computer program product of claim 1, wherein the one or more instructions, when executed by at least one processor of the fluid injector system, further cause the at least one processor to: during the second phase of the injection protocol, adjust a speed of the first drive component to inject the first fluid at a flow rate to reach the desired steady-state ratio of the first fluid and the second fluid through the fluid conduit during the second phase of the injection protocol.

6. The computer program product of claim 1, wherein the fluid injector system further comprises at least a first valve controlling fluid communication between the fluid conduit and a first reservoir containing the first fluid, and a second valve controlling fluid communication between the second reservoir and the fluid conduit, and
wherein the one or more instructions, when executed by at least one processor of the fluid injector system, further cause the at least one processor to close the second valve prior to pressurizing the second fluid relative to the pressure of the first fluid in the fluid conduit.

7. The computer program product of claim 6, wherein the one or more instructions, when executed by at least one processor of the fluid injector system, further cause the at least one processor to: open the second valve isolating the second fluid reservoir from the first fluid reservoir and the fluid conduit during a transition from the first phase of the injection protocol to the second phase of the injection protocol.

8. The computer program product of claim 1, wherein pressurizing the second fluid relative to the pressure of the first fluid comprises selecting a pressure ratio of the pressure of the first fluid and a pressure of the second fluid to reach the desired steady-state flow ratio of the first fluid and the second fluid for the second phase.

9. The computer program product of claim 1, wherein pressurizing the second fluid relative to the pressure of the first fluid comprises gradually increasing a pressure of the second fluid such that, at a transition from the first phase of the injection protocol to the second phase of the injection protocol, the pressure of the second fluid is substantially equal to the pressure of the first fluid.

10. The computer program product of claim 1, the one or more instructions, when executed by at least one processor of the fluid injector system, further cause the at least one processor to: during the first phase of the injection protocol prior to transitioning to the second phase of the injection protocol, adjusting at least one property of the injection protocol to prevent backflow of the first fluid into the second fluid reservoir.

11. The computer program product of claim 10, wherein adjusting at least one property of the injection protocol comprises at least one of: advancing the second drive component at a constant speed, linearly increasing a speed of the second drive component, and exponentially increasing the speed of the second drive component.

12. A method of performing an injection protocol comprising at least a first phase and a second phase using a fluid injector system, the second phase subsequent to the first phase, the method comprising:
providing a control device operatively associated with a first drive component and a second drive component, the first drive component configured to pressurize and inject a first fluid from a first fluid reservoir through a fluid conduit, the second drive component configured to pressurize and inject a second fluid from a second fluid reservoir through the fluid conduit,
actuating at least one of the first drive component and the second drive component to inject at least one of the first fluid and the second fluid through the fluid conduit at a first desired steady-state ratio during the first phase of the injection protocol;
actuating the first drive component and the second drive component to inject the first fluid and the second fluid through the fluid conduit such that a volumetric ratio of the second fluid displaced from the second fluid reservoir relative to the first fluid displaced from the first fluid reservoir exceeds a second desired steady-state ratio during an initial portion of the second phase of the injection protocol; and
actuating at least one of the first drive component and the second drive component to reduce the volumetric ratio until the volumetric ratio reaches the second desired steady-state ratio of the first fluid and the second fluid in the second phase subsequent to the initial portion of the second phase.

13. The method of claim 12, wherein the first fluid comprises a contrast medium and the second fluid comprises a diluent.

14. The method of claim 12, wherein the second desired steady-state ratio of the first fluid and the second fluid in the second phase is reached at a quicker rate than if the actuation of the first drive component and the second drive component to inject the first fluid and the second fluid through the fluid conduit such that a volumetric ratio of the second fluid displaced from the second fluid reservoir relative to the first fluid displaced from the first fluid reservoir does not exceed a second desired steady-state ratio.

15. The method of claim 12, further comprising ceasing actuation or reduce a speed of the first drive component during the first phase of the injection protocol prior to transitioning to the second phase of the injection protocol.

16. The method of claim 12, wherein the fluid injector system further comprises at least a first valve controlling fluid communication between the first reservoir and the fluid conduit and a second valve controlling fluid communication between the second reservoir and the fluid conduit, and
wherein the method further comprises opening the second valve isolating the second drive component from the first fluid reservoir and the fluid conduit during a transition from the first phase of the injection protocol to the second phase of the injection protocol.

17. The method of claim 12, further comprising actuating the second drive component to pressurize the second fluid relative to a pressure of the first fluid in the fluid conduit during the first phase of the injection protocol prior to transitioning to the second phase of the injection protocol.

18. The method of claim 17, wherein the fluid injector system further comprises at least a first valve controlling fluid communication between the first reservoir and the fluid conduit and a second valve controlling fluid communication between the second reservoir and the fluid conduit, and
wherein the method further comprises closing the second valve prior to pressurizing the second fluid relative to the pressure of the first fluid in the fluid conduit.

19. The method of claim 12, further comprising adjusting at least one property of the injection protocol to prevent backflow of the first fluid into the second fluid reservoir during the first phase of the injection protocol prior to transitioning to the second phase of the injection protocol.

20. The method of claim 19, wherein adjusting at least one property of the injection protocol comprises at least one of: advancing the second drive component at a constant speed, linearly increasing a speed of the second drive component, and exponentially increasing the speed of the second drive component.
